(19) United States
(12) Patent Application Publication
(10) Pub. No.: US 2006/0035373 A1
(43) Pub. Date: Feb. 16, 2006

(54) Stem cell libraries

(76) Inventors: Hongbing Zhang, Albany, CA (US); Lewis Thomas Williams, Mill Valley, CA (US); Keting Chu, Woodside, CA (US); Fred Cohen, San Francisco, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/003,840

(22) Filed: Dec. 6, 2004

Related U.S. Application Data
(62) Division of application No. 10/516,605, filed on Jun. 3, 2005.
(60) Provisional application No. 60/423,041, filed on Nov. 1, 2002.
(60) Provisional application No. 60/454,576, filed on Mar. 13, 2003.

(51) Int. Cl. C12N 5/08; C12N 15/87; C40B 40/02

(52) U.S. Cl. 435/366; 435/455

(57) ABSTRACT

A stem cell library is created by genetically modifying stem cells with nucleic acids encoding polypeptides which can promote stem cell differentiation into specific cell types. Alternatively, the stem cell library is exposed to an externally added factor that promotes stem cell differentiation into a desired cell line, e.g., neuronal or muscle. The library is used to determine the effect of the encoded protein on the differentiation process. The library is also used to produce nucleic acids for insertion into embryonic stem cells to produce transfected embryonic stem cells. The nucleic acids are inserted into a locus that permits widespread expression of the encoded polypeptide in animals produced from blastocysts that incorporate the transfected cells. Non-human chimeric animals produced by combining blastocysts derived from animal models of human disease and embryonic stem cells transfected with molecules from the library provide an in vivo system for therapeutic design.

### PRIORITY CLAIM

This application claims priority to provisional applications 60/423,041, Stem Cell Library, filed in the U.S. Patent and Trademark Office Nov. 1, 2002 and 60/454,576, Stem Cell Library, filed in the U.S. Patent and Trademark Office Mar. 13, 2003, both of which are incorporated by reference in their entireties.

### TECHNICAL FIELD

The present invention pertains generally to the fields of biology, pharmaceuticals and medicine. In particular, the invention relates to the use of cell libraries to study gene or protein function.

### BACKGROUND ART

With the completion of the sequencing of the human genome and the genomes of certain other organisms, there is now a plethora of novel genes and proteins of unknown function. In the past, scientists have isolated new genes one by one and have studied the function of the resulting proteins one by one. This approach, however, is not conducive to the large scale study of gene or protein function. There is, thus, a need for novel methodologies for massively parallel study to enable rapid understanding of gene and protein functions, whether gain of function or loss of function, and rapid discovery of novel molecules that are useful as therapeutics.

Libraries of cells with heterologous nucleic acids that produce polypeptides can address this need. However, presently available libraries are not well defined in terms of their components, do not contain equal representation of each component, and are not enriched in molecules belonging to a particular class of interest.

### SUMMARY OF THE INVENTION

Stem cells are transfected with heterologous nucleic acids that express heterologous polypeptides. The stem cells have the capacity to differentiate into a plurality of cell types that express the heterologous polypeptides. The stem cells can be incorporated into a blastocyst which develops into a chimeric embryo, fetus, or adult non-human animal. When the heterologous nucleic acid is introduced into the stem cell at certain loci, e.g., the ROSA 26 locus, the chimeric non-human animal expresses the heterologous polypeptide in most or all of its tissues. Animals expressing such heterologous polypeptides provide in vivo models to study the effect of genes and proteins associated with the expressed polypeptides in the animal. When the stem cells are incorporated into a blastocyst of an animal model of a human disease, the effect of the transfected nucleic acids and the polypeptides they express on the disease can be assessed by observing the phenotype of the animal. Libraries of the stem cells can be compiled that express selected polypeptides known or hypothesized to modulate selected in vivo or in vitro functions, and can be used to screen, test, or compare potentially therapeutic or otherwise modulatory agents.

### BRIEF DESCRIPTION OF THE DRAWINGS

FIG. 1 is a schematic representation depicting an example of the generation of a targeting vector for a secreted factor. It is described in more detail in Example 1.

FIG. 2 illustrates the expression of a transmembrane protein, the EGF receptor, on the cell surface of several embryonic stem cell clones. Six positive clones expressed EGF receptor on the cell surface, as demonstrated by Western blot in the left panel and by fluorescence activated cell sorting (FACS) in the right panel. The Western blot shows the immunoreactivity of EGF receptor in cellular lysates. Lane 1 contained molecular weight markers. Lane 2 contained 40 μg clone 13 lysate. Lane 3 contained 40 μg clone 14 lysate. Lane 4 contained 40 μg clone 38 lysate. Lane 5 contained 40 μg clone 64 lysate. Lane 6 contained 40 μg clone 75 lysate. Lane 7 contained 40 μg clone 86 lysate. Lane 8 was a negative control lane. Lanes 9-11 contained 5, 10, and 20 μg A431 cell lysate respectively, as positive controls. The FACS analysis demonstrates that the EGF receptor was expressed on the surface of clone 13 cells.

FIG. 3 illustrates the functionality of proteins expressed by embryonic stem cells. FIG. 3A shows the appearance of the differentiation marker CD235 in human bone marrow CD34+ cells in response to commercially available recombinant erythropoietin (Epo). The left panel shows the FACS profile of the cells in the absence of Epo. The right panel shows the FACS profile of the cells in the presence of Epo. FIG. 3B shows the appearance of the differentiation marker CD235 in human bone marrow CD34+ cells in response to Epo expressed and secreted by the embryoid body of the invention. The left panel shows the FACS profile of the cells in the presence of the negative control IL-5, which was expressed and secreted from the embryoid body of the invention, but which does not induce differentiation of CD34+ cells. The right panel shows the FACS profile of the cells in the presence of Epo expressed and secreted from the embryoid body of the invention. The expressed, secreted EPO induced the appearance of the differentiation marker CD235 in CD34+ cells. The percent differentiated cells is shown in each of the four panels. FIG. 3C shows the FACS profile of the TER 119 marker in the CD34+ cells, confirming that the cells are of human origin.

FIG. 4 illustrates the ability of proteins expressed and secreted from embryonic stem cells to induce mitosis in TF-1 cells. The dark bars show the mitogenic activity of commercially available recombinant IL-5 and Epo. The light bars show the mitogenic activity of conditioned medium from embryonic stem cells secreting IL-5 and Epo.

### DETAILED DESCRIPTION OF THE INVENTION

Definitions

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information contained in a gene into a gene product. A gene product can be the direct transcriptional product of a gene (e.g., mRNA, tRNA, rRNA, antisense RNA, ribozyme, structural RNA, or any other type of RNA) or a protein produced by translation of an mRNA. Gene products also include RNAs which are modified, e.g., by processes such as capping, polyadenylation, methylation, and editing, and proteins which are modified by, e.g., methylation, acetylation, phosphorylation, ubiquitination, ADP-ribosylation, myristilation, prenylation, and glycosylation.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from viral, prokaryotic, or eucaryotic mRNA, genomic DNA sequences from viral (e.g. DNA viruses and retroviruses) or prokaryotic DNA, and synthetic DNA sequences. A transcription termination sequence may be located 3′ to the coding sequence.

A “nucleic acid” molecule can include both double- and single-stranded sequences and refers to, but is not limited to, cDNA from viral, prokaryotic or eucaryotic mRNA, genomic DNA sequences from viral (e.g. DNA viruses and retroviruses) or prokaryotic DNA, and synthetic DNA sequences. The term also captures sequences that include any of the known base analogs of DNA and RNA.

A “vector” is a polynucleotide construct comprising an expression cassette, a wide variety of which are known in the art. Vectors include, but are not limited to, plasmids; cosmids; viral vectors; human, yeast, bacterial, P1-derived artificial chromosomes (HAC's, YAC's, BAC's, PAC's, etc.), and mini-chromosomes. Vectors can provide for nucleic acid expression, for nucleic acid propagation, or both. A recombinant vector or construct that includes a nucleic acid of the invention is useful for propagating a nucleic acid in a host cell; such vectors are known as “cloning vectors.” Vectors can transfer nucleic acid between host cells derived from disparate organisms; these are known in the art as “shuttle vectors.” Vectors can also insert a subject nucleic acid into a host cell's chromosome; these are known in the art as “insertion vectors.” Vectors can express either sense or antisense RNA transcripts of the invention in vitro (e.g., in a cell-free system or within an in vitro cultured host cell) or in vivo (e.g., in a multicellular plant or animal); these are known in the art as “expression vectors,” which can be part of an expression system.

Vectors typically include at least one origin of replication, at least one site for insertion of heterologous nucleic acid (e.g., in the form of a polylinker with multiple, tightly clustered, single cutting restriction endonuclease recognition sites), and at least one selectable marker, although some integrative vectors will lack an origin that is functional in the host to be chromosomally modified, and some vectors will lack selectable markers.

“Retroviruses” are a class of enveloped viruses containing a single stranded RNA molecule as the genome. Retroviral vectors are frequently used for or gene therapy, because of their ability to integrate into the cellular genome (Jolly (1994) Cancer Gene Ther. 1:51-64 and Hodgson (1995) BioTechnology 13:222-225). Retroviral vectors can be based upon the Moloney murine leukemia virus (Mo-MLV). Mo-MLV is an amphotrophic virus, capable of infecting both mouse cells and human cells. The viral genes are replaced with the transgene of interest and expressed on plasmids in the packaging cell line.

“Adenoviruses” are non-enveloped viruses containing a linear double stranded DNA genome. The life cycle does not normally involve integration into the host genome, rather adenoviruses replicate as episomal elements in the nucleus of the host cell. Adenovirus-based vectors offer several unique advantages, including tropism for both dividing and non-dividing cells, minimal pathogenic potential, ability to replicate to high titer for preparation of vector stocks, and the potential to carry large inserts (Berkner (1992) Curr. Top. Micro. Immunol. 158: 39-66 and Jolly (1994) Cancer Gene Therapy 1:51-64).

“Adeno-associated viruses” (AAV) are non-pathogenic human parvoviruses, dependent on a helper virus to proliferate. AAV are capable of infecting both dividing and non dividing cells, and in the absence of a helper virus integrate into a specific point of the host genome at a high frequency. Recombinant AAV can also efficiently integrate into the host genome, can transduce non-dividing cells, and does not induce an immune response which destroys the transformed cells.

A “promoter” as used herein is a DNA regulatory region capable of binding RNA polymerase in a mammalian cell and initiating transcription of a downstream (3′ direction) coding sequence operably linked thereto. For purposes of the present invention, a promoter sequence includes the minimum number of bases or elements necessary to initiate transcription of a gene of interest at levels detectable above background. Within the promoter sequence is a transcription initiation site, as well as protein binding domains (consensus sequences) responsible for the binding of RNA polymerase. Eucaryotic promoters will often, but not always, contain “TATA” boxes and “CAT” boxes.

Some promoters are “constitutive,” and direct transcription in the absence of regulatory influences. Some promoters are “tissue specific,” and initiate transcription exclusively or selectively in one or a few tissue types. Some promoters are “inducible,” and achieve gene transcription under the influence of an inducer. Induction can occur, e.g., as the result of a physiologic response, a response to outside signals, or as the result of artificial manipulation. Some promoters respond to the presence of tetracycline; “rtTA” is a reverse tetracycline controlled transactivator.

“Operably linked” refers to an arrangement of elements wherein the components so described are configured so as to perform their desired function. Thus, a given promoter operably linked to a coding sequence is capable of effecting the expression of the coding sequence when the proper transcription factors, etc., are present. The promoter need not be contiguous with the coding sequence, so long as it functions to direct the expression thereof. Thus, for example, intervening untranslated yet transcribed sequences can be present between the promoter sequence and the coding sequence, as can translated introns, and the promoter sequence can still be considered “operably linked” to the coding sequence.

A “control element” refers to a polynucleotide sequence which aids in the expression of a coding sequence to which it is linked. The term includes promoters, transcription termination sequences, upstream regulatory domains, polyadenylation signals, and when appropriate, leader sequences and enhancers, which collectively provide for the transcription and translation of a coding sequence in a host cell.

By “selectable marker” is meant a gene which confers a phenotype on a cell expressing the marker, such that the cell can be identified under appropriate conditions. Generally, a selectable marker allows selection of transformed cells based on their ability to thrive in the presence or absence of a chemical or other agent that inhibits an essential cell function. Suitable markers, therefore, include genes coding for proteins which confer drug resistance or sensitivity thereto, impart color to, or change the antigenic characteristics of those cells transfected with a molecule encoding the selectable marker, when the cells are grown in an appropriate selective medium. For example, selectable markers include cytotoxic markers and drug resistance markers, whereby cells are selected by their ability to grow on media containing one or more of the cytotoxins or drugs; auxotrophic markers by which cells are selected by their ability to grow on defined media with or without particular nutrients or supplements, such as thymidine and hypoxanthine; metabolic markers by which cells are selected for, e.g., their ability to grow on defined media containing the appropriate sugar as the sole carbon source; and markers which confer the ability of cells to form colored colonies on chromogenic substrates or cause cells to fluoresce.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide.

A “locus” is the position of a DNA segment, e.g., a gene, on a chromosome. The “ROSA 26” locus is the position at which the ROSAβgeo retrovirus integrated into the genome of the ROSAβgeo26 (ROSA26) mutant strain of mice. It maps to mouse chromosome 6 (Zambrowicz et al., 1997). The ROSA26 mouse strain was produced by random retroviral gene trapping in embryonic stem cells. Gene traps use vectors to identify genes that exhibit discrete patterns of expression during development and differentiation. The trap vectors contain a reporter gene that is not expressed unless it integrates into an intron or exon of a transcription unit. Integration results in an expression pattern that reflects the pattern of the endogenous transcription unit. The reporter gene provides a molecular tag for cloning the trapped gene. The ROSA26 cell line is a mouse gene trap line derived from the ROSA26 mouse strain that displays ubiquitous expression of the reporter gene during embryonic development. The reporter gene in the ROSA26 mouse strain and cell line is β-galactosidase.

The “ROSA 5” locus is the position at which the ROSAβgeo retrovirus integrated into the genome of the ROSAβgeo5 (ROSA5) mutant strain of mice. The “ROSA 11” locus is the position at which the ROSAβgeo retrovirus integrated into the genome of the ROSAβgeo11 (ROSA11) mutant strain of mice. The “G3BP(BT5) locus” is the position of a phosphorylation-dependent endoribonuclease that interacts with RasGAP.

A “tumor suppressor” gene is a gene that can reverse the effect of a gene or other agent that promotes tumor formation. For example, a tumor suppressor gene may reverse the effect of a mutation that promotes tumor formation.

“Transfected” means with introduced DNA or RNA, with or without the use of any accompanying facilitating agents, e.g., lipofectamine. Methods for transfection that are known in the art include calcium phosphate transfection, DEAE dextran transfection, protoplast fusion, electroporation, and lipofection.

“Transgene” means a nucleic acid sequence that is incorporated into a transgenic organism. A “transgene” can contain one or more transcriptional regulatory sequences, and other sequences, such as introns, that may be useful for expressing or secreting the nucleic acid or fusion protein it encodes.

“Transformation,” as used herein, refers to the insertion of an exogenous polynucleotide into a host cell, irrespective of the method used for insertion: for example, transformation by direct uptake, transfection, infection, and the like. The exogenous polynucleotide may be maintained as a nonintegrated vector, for example, an episome, or alternatively, may be integrated into the host genome.

An “episome” is a sequence of DNA or RNA that can exist as free, autonomously replicating nucleic acid or be attached to and integrated into the chromosome of the cell, in which case it replicates along with the chromosome. Examples of episomes include bacteriophages and the male sex factor of E. coli.

“Polyoma large T antigen” is a protein translated from an mRNA generated by alternative splicing of the primary transcript encoded by the polyoma virus early region. Polyoma virus DNA replicates as free, unintegrated mini-chromosomes. Three related proteins encoded by the early region are expressed shortly after infection, the large tumor (T) antigen, middle T antigen and small T antigen. Large T antigen typically is required for initiating viral DNA replication.

An “origin of replication” is the sequence of DNA at which DNA replication begins. Replication is generally controlled at the point of initiation. “PyF101” is an enhancer in the polyoma virus origin of replication.

An “interfering RNA” (RNAi) molecule is an RNA molecule that partially or completely silences one or more eukaryotic genes. For example, double stranded RNA can induce the homology-dependent degradation of its cognate mRNA. Use of RNAi to reduce a level of a particular mRNA and/or protein is based on the interfering properties of double-stranded RNA derived from the coding regions of a gene. The technique can reduce the time between identifying an interesting gene sequence and understanding its function, and thus is an efficient high-throughput method for disrupting gene function. RNAi can also help identify the biochemical mode of action of a drug and identify other genes encoding products that can respond or interact with specific compounds.

The terms “polypeptide” and “protein” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include post-expression modifications of the poiypeptide, for example, glycosylation, acetylation, phosphorylation, and the like. Furthermore, for purposes of the present invention, a “polypeptide” refers to a protein which includes modifications, such as deletions, additions and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

A “library” of polynucleotides comprises a collection of sequence information of a plurality of polynucleotide sequences, which information is provided in either biochemical form (e.g., as a collection of polynucleotide molecules), or in electronic form (e.g., as a collection of polynucleotide sequences stored in a computer-readable form, as in a computer-based system, a computer data file, and/or as part of a computer program).

A “library” of polypeptides comprises a collection of sequence information of a plurality of polypeptide sequences, which information is provided in, e.g., a collection of polypeptide sequences stored in a computer-readable form, as in a computer-based system, a computer data file, and/or as part of a computer program.

By “isolated” is meant, when referring to a polypeptide, that the indicated molecule is separate and discrete from the whole organism with which the molecule is found in nature or is present in the substantial absence of other biological macromolecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

A “fragment” of a polypeptide or protein is a polypeptide consisting of only a part of the intact full-length polypeptide sequence and structure. The fragment can include a C-terminal deletion, an N-terminal deletion, and/or an internal deletion of the native polypeptide. A fragment of a protein may include at least about 5-10 contiguous amino acid residues of the full-length molecule, at least about 15-25 contiguous amino acid residues of the full-length molecule, at least about 20-50 or more contiguous amino acid residues of the full-length molecule, or any integer between 5 amino acids and the full-length sequence.

An “active” fragment is one having structural, regulatory, or biochemical functions of a naturally occurring molecule or any function related to or associated with a metabolic or physiological process. The activity can include an improved desired activity, or a decreased undesirable activity. For example, an entity demonstrates activity when it participates in a molecular interaction with another molecule, or when it has therapeutic value in alleviating a disease condition, or when it has prophylactic value in inducing an immune response to the molecule, or when it has diagnostic value in determining the presence of the molecule, such as an active fragment of a polynucleotide that can be detected as unique for the polynucleotide molecule, or that can be used as a primer in PCR.

“Homology” refers to the percent identity between two polynucleotide or two polypeptide moieties. Two DNA, or two polypeptide sequences are “substantially homologous” to each other when the sequences exhibit at least about 50%, at least about 75%, at least about 80%-85%, at least about 90%, or at least about 95%-98% sequence identity over a defined length of the molecules. As used herein, “substantially homologous” also refers to sequences showing complete identity to the specified DNA or polypeptide sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National Biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

“Secreted proteins,” also referred to as secreted factors, are proteins that are produced by cells and exported extracellularly, extracellular fragments of transmembrane proteins that are proteolytically cleaved, and extracellular fragments of cell surface receptors, which may be soluble. Secreted proteins mediate many and widely variant biological functions. They can act as ligands for binding to receptors on cell surfaces in ligand/receptor interactions, and trigger intracellular responses, such as inducing signal transduction, inducing cellular growth, proliferation, or differentiation, or inducing production of other factors that, in turn, mediate such activities.

“Transmembrane proteins” extend into or through the cell membrane's lipid bilayer; they can span the membrane once, or more than once. Transmembrane proteins that span the membrane once are “single transmembrane proteins” (STM), and transmembrane proteins that span the membrane more than once are “multiple transmembrane proteins” (MTM). Examples of transmembrane proteins include the insulin receptor, adenylate cyclase, and intestinal brush border esterase.

A single transmembrane protein typically has one transmembrane (TM) domain, spanning a series of consecutive amino acid residues. A multi-transmembrane protein typically has more than one TM domain, each spanning a series of consecutive amino acid residues.

A “kinase” is an enzyme that catalyzes the transfer of phosphate groups from phosphate donors to acceptor substrates. Kinase substrates include, but are not limited to, proteins and lipids. A “phosphatase,” as indicated above, is an enzyme that catalyses the hydrolysis of esters of phosphoric acid. Its substrates include, but are not limited to, nucleic acids, proteins, and lipids.

Kinases and phosphatases are counteracting: kinases add phosphate groups and phosphatases liberate phosphate groups. The counteracting activities of kinases and phosphatases provide cells with a “switch” that can turn on or turn off the function of various proteins. The activity of any protein regulated by phosphorylation depends on the balance, at any given time, between the activities of the kinase(s) that phosphorylate it, and the phosphatase(s) that dephosphorylate it. Phosphorylation plays a important role in intercellular communication during development, homeostasis, and the function of major bodily systems, including the immune system. In conjunction, kinases and phosphatases control such diverse and essential cellular processes as transcription, cell division, cell cycle progression, differentiation, cytoskeletal function, apoptosis, receptor function, learning and memory, hematopoeisis, fertilization, neural transmission, muscle contraction, non-muscle motor function, glycogen metabolism, and hormone secretion.

“Proteases,” also known as endopeptidases, are enzymes that cleave polypeptide chains by hydrolyzing peptide bonds at positions within the amino acid chain. Different proteases recognize different polypeptide sequences. Endopeptidase substrate specificities vary from broad to narrow; for example, subtilisins are relatively non-specific, and can cleave polypeptide chains with a wide variety of amino acid sequences, whereas thrombin is more specific and can only cleave polypeptide chains with an arginine residue on the carboxyl side of the susceptible peptide bond and glycine on the amino side.

“Phosphodiesterases” are enzymes that cleave phosphodiester bonds, i.e., bonds formed by two hydroxyl groups in an ester linkage to the same phosphate group, such as those between adjacent RNA or DNA nucleotides. Phosphodiesterases are found in both soluble and membrane-associated forms. Most phosphodiesterases act within a network of signal transduction molecules and other signaling effectors, and are modulated by components of these pathways. Phosphodiesterases regulate the metabolism and synthesis of cyclic nucleotides in signal-transduction pathways. They hydrolyze cAMP and cGMP, molecules that play an important and widespread role in signal transduction. Phosphodiesterases also repair damage to nucleic acids. Some phosphodiesterases are regulated primarily by calcium and calmodulin, others are regulated primarily by cGMP. They differ in their sensitivity to individual inhibitors, but share a homologous catalytic region.

Cells transport proteins and organelles in an orderly and regulated manner along cytoskeletal filaments. “Kinesins” are molecular motor proteins that can carry such cargo along the cytoskeletal filaments to specific destinations, in a regulated manner. Exemplary membrane-bound cargoes include mitochondria, lysosomes, endoplasmic reticulum, and axonal vesicles; exemplary nonmembranous cargoes include mRNAs, tubulin monomers, and intermediate filaments.

“Hormone receptors” are polypeptides that bind to a specific hormone and initiate a cellular response. They can be present on the cell surface or inside the cell. Protein hormone receptors are generally present on the cell plasma membrane, with the ligand binding site on an extracellular domain. Nuclear hormone receptors generally function by crossing the plasma membrane of target cells and binding to intracellular protein ligands. Ligand binding activates these receptors in some instances, exposing a DNA binding domain which regulates the transcription of specific genes. Generally, nuclear hormone receptors bind to specific DNA sequences adjacent to or in the vicinity of the genes regulated by their ligand. A multitude of cell type-specific regulatory proteins can collaborate with the nuclear hormone receptor to influence the transcription of specific genes or sets of genes. Examples of nuclear hormone receptors include estrogen-related receptors, such as hERR1, which modulates the estrogen receptor-mediated response of the lactoferrin gene promoter and is a transcriptional regulator of the human medium chain acyl coenzyme A dehydrogenase gene. Examples of nuclear hormone receptors also include photoreceptor-specific nuclear receptors, such as NR2E3, which are part of a large family of nuclear receptor transcription factors involved in signaling pathways.

A “histone deacetylase” is an enzyme that removes acetyl groups from histones, which are basic proteins found in the cell nucleus. Histone deacetylases play a role in the post-synthetic structural modification of histones, and contribute to the control of chromatin structure and function. They can, e.g., remove the acetyl group from the epsilon-amino group of a lysine residue. Histone deacetylase inhibitors have utility as anticancer agents due to their ability to cause growth arrest, terminal differentiation and/or apoptosis in carcinoma cells.

A “ubiquitin E3 ligase” is a ubiquitin protein ligase that is a component in the pathway that attaches ubiquitin to specific proteins, designating them for degradation. For example, a multi-subunit E3 ubiquitin ligase targets the hypoxia-inducible transcription factor Hiflalpha for proteasomal degradation under conditions of normal oxygenation.

A “growth factor” is an extracellular polypeptide signaling molecule that stimulates a cell to grow or proliferate. Many types of growth factors exist, including protein hormones and steroid hormones. Some growth factors have a broad specificity, and some have a narrow specificity. Examples of growth factors with broad specificity include platelet-derived growth factor (PDGF), epidermal growth factor, insulin like growth factor 1, transforming growth factorβ, and fibroblast growth factor, which act on many classes of cells. Examples of growth factors with narrow specificity include erythropoietin, which induces proliferation of precursors of red blood cells, interleukin-2, which stimulates proliferation of activated T-lymphocytes, interleukin-3, which stimulates proliferation and survival of various types of blood cell precursors, and nerve growth factor, which promotes the survival and the outgrowth of nerve processes from specific classes of neurons. Other examples of growth factors include keratinocyte growth factor (KGF), brain-derived neurotrophic factor (BDNF), granulocyte colony-stimulating factor (G-CSF), and granulocyte-macrophage colony-stimulating factor (GM-CSF).

Most growth factors have other actions in addition to inducing cell growth or proliferation, e.g., they may influence survival, differentiation, migration, or other cellular functions. Growth factors can have complex effects on their targets, e.g., they may act on some cells to stimulate cell division, and on others to inhibit it. They may stimulate growth at one concentration, and inhibit it an another. Growth factors are also involved in tumorigenesis.

Keratinocyte growth factor (KGF) stimulates the growth of keratinocytes, and is useful for repairing tissue after chemotherapy or radiotherapy.

A “cytokine” is an extracellular signaling protein or peptide that acts as a local mediator in communication among cells. Cytokines regulate proliferation and differentiation, for example, they mediate differentiation of cells in the hematopoeitic lineage. Examples of cytokines include interleukins, interferons, and colony stimulating factors of the hematopoeitic system. Some cytokines, e.g., interferons and interleukins, can be induced by viral activity, and possess antiviral activity.

Transforming growth factor-betas (TGF-β) regulate pivotal cellular processes such as proliferation, differentiation, and apoptosis. These ligands bind transmembrane serine/threonine kinase receptors. TGF-β receptors initiate distinct signaling cascades by varying their cellular distribution, oligomerization mode, and formation of complexes with different cell surface receptors. For example, the type I receptor phosphorylates the intracellular Smad protein effectors, which upon oligomerization enter the nucleus to regulate transcription following their assembly with transcriptional co-factors and co-modulators. The broad array of intracellular proteins that influence TGF-β pathways demonstrates that TGF-β signal transduction is not linear but rather comprises a complex network of cascades that mutually influence one other.

Transforming growth factor-betas act as tumor suppressors in the breast, and the loss of TGFβ receptors seen in some human breast hyperplasia plays a causal role in breast tumor development (Wakefield et al., 2000). Overexpressing TGFβ can suppress tumorigenesis in the mammary gland. Conversely, loss of TGFβ response increases spontaneous and induced mammary gland tumorigenesis. Genetically altered mouse models in current existence provide tools to analyze TGFβ action in the context of the whole animal, permitting the development of pharmacologic agents to treat and prevent cancer.

“Wnt proteins” are signaling molecules that are generally secreted. They adhere to the plasma membrane of the cells from which they are secreted, and thus, are likely to signal over relatively short distances from their origin. Wnt proteins are ligands for receptors with seven transmembrane regions that comprise the “frizzled” gene family. Wnt protein ligands bind frizzled receptors, resulting in the generation of an intracellular signal. This signal can diversify into at least three interconnecting pathways. There is cross-talk among these three Wnt pathways, and these three pathways can also interact with other, non-Wnt, signaling pathways. During development, Wnt proteins play diverse roles in governing cell proliferation, migration, polarity, and death. Wnt proteins are involved in establishing body axis formation, such as the polarity of insect and vertebrate limbs, stem cell renewal and differentiation, and development of many organs, e.g., the urogenital system in sex determination, neural tissues, lung, and muscle. In adults, Wnt proteins function in homeostasis.

“Amyloid-beta peptide” (Aβ) is a major component of amyloid in the brains of Alzheimer's disease patients. The beta-amyloid precursor protein is cleaved to form amyloid beta-peptides, which are insoluble. Polymerization of the amyloid beta-peptide and deposition of neurofibrillary tangles and senile plaques largely comprised of amyloid beta-peptide is a feature of the pathogenesis of Alzheimer's disease. Inhibiting the formation of toxic polymers of amyloid beta-peptide has emerged as an approach to developing therapeutics.

“Notch” genes are neurogenic genes, first described in Drosophila. The function of Notch gene products to prevent ectodermal cells from differentiating into neuroblasts.

A “co-factor” is a molecule that acts in concert with another substance to bring about certain effects.

Rat mesenchymal stem cells have been genetically engineered using ex vivo retroviral transduction to overexpress the prosurvival gene “Akt1,” a protein kinase that is the product of the v-aKt oncogene (Mangi et al., 2003).

A “lymphokine” is a cytokine produced by a leukocyte, which acts upon another cell. Examples include interleukins, interferon-alpha, tumor necrosis factor-alpha, and granulocyte/monocyte colony-stimulating factor.

An “anti-inflammatory molecule” is a molecule that can diminish, eliminate, or prevent a response to injury or infection. For example, an antihistamine can counteract the effect of the inflammatory mediator histamine.

An “anti-cancer molecule” is a molecule that can diminish, eliminate, or prevent the effects of cancer. It includes pharmaceuticals and antibodies.

An “apoptotic molecule” is a molecule that induces a cell to move towards apoptosis, or programmed cell death. Normally functioning cells undergo apoptosis when their age or their state of health so dictates. Apoptosis is an active process requiring metabolic activity by the dying cell, often characterized by cleavage of the DNA into fragments. Cells that die by apoptosis do not generally elicit the inflammatory response associated with necrosis. Cancer cells do not typically undergo normal apoptosis.

A first and second therapeutic molecule working in “conjunction” work in association with one another to achieve a therapeutic effect.

A first and second heterologous nucleic acid sequence that “interact” with one another have an effect on one another such that one of the molecules influences the other. Either may act upon the other, or both may act upon each other.

A “therapeutic factor” encoded by a first heterologous nucleic acid sequence of a modified mesenchymal cell is a factor, excluding a cell survival factor (Mangi et al., (2003) Nat. Med. 9:1195-1201; WO 03/073998), that is preventative, palliative, curative, or otherwise useful in treating or ameliorating, or preventing the recurrence of a disease, disorder, syndrome or condition.

“Telomerase” is a DNA polymerase enzyme that selectively elongates DNA from the telomere, i.e., the end of a chromosome. Telomeric DNA contains multiple, e.g., hundreds, of tandem repeats of a hexanucleotide sequence. One strand of telomeric DNA is G-rich at the 3′ end, and slightly longer than the other strand. Telomeric DNA can form large duplex loops, wherein the single-stranded region at the very end of the structure loops back to form a DNA duplex with another part of the repeated sequence, displacing a part of the original telomeric duplex. This loop-like structure is formed and stabilized by specific telomere-binding proteins. These structures protect and mask the end of the chromosome.

The telomeric loop-like structures are generated by telomerase. The telomerase enzyme contains an RNA molecule that serves as the template for elongating the G-rich strand of telomeric DNA. Thus, the enzyme carries the information necessary to generate the telomere sequences. Telomerases also have a protein component, which is related to reverse transcriptases. Telomerases can influence cell aging, and play a role in cellular cancer biology.

“Tumor necrosis factor” (TNF) encompasses a family of receptor ligands that display pleiotropic effects on normal and malignant cells. Natural induction of TNF is protective, but its overproduction may be detrimental and even lethal to the host. TNF elicits a variety of responses in different cell types. TNF was originally characterized as an antitumor agent and a cytotoxic factor for malignant cells. It subverts the electron transport system of mitochondria to produce oxygen radicals, which can kill malignant cells lacking protective enzymes. TNF also plays a role in the defense against viral, bacterial, and parasitic infections, and in mediating autoimmune responses (Fiers, (1991) FEBS Lett. 285:199-212). TNF inhibitors have been used to treat psoriasis (Weinberg and Saini, (2003) Cutis. 71:25-29). It is synonymous with “cachectin.”

An “immunological response” to a composition, including a transformed stem cell population or fragments of a transformed stem cell, is the development in the host of a cellular and/or antibody-mediated immune response to the composition. Usually, an “immunological response” includes, but is not limited to, the production of antibodies, B cells, helper T cells, suppressor T cells, and/or cytotoxic T cells and/or γδT cells, directed specifically to an antigen or antigens included in the composition.

The terms “immunogenic” protein or polypeptide refer to an amino acid sequence which elicits an immunological response. An “immunogenic” protein or polypeptide, as used herein, includes the full-length sequence of the protein in question, including precursor and mature forms, analogs thereof, or immunogenic fragments thereof.

By “immunogenic fragment” is meant a fragment of a protein which includes one or more epitopes and thus elicits the immunological response described above. Such fragments can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by, e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al., Proc. Natl. Acad. Sci. USA (1981) 78:3824-3828 for determining antigenicity profiles, and the Kyte-Doolittle technique, Kyte et al., J. Mol. Biol. (1982) 157:105-132 for determining hydropathy plots.

Immunogenic fragments, for purposes of the present invention, will usually be at least about 2 amino acids in length, about 5 amino acids in length, or at least about 10 to 15 amino acids in length. There is no upper limit to the length of the fragment, which could comprise nearly the full-length of the protein sequence, or a fusion protein comprising two or more epitopes of the protein in question.

The term “antibody” encompasses polyclonal and monoclonal antibody preparations, as well as preparations including hybrid antibodies, altered antibodies, chimeric antibodies, and humanized antibodies, as well as hybrid (chimeric) antibody molecules (see, for example, Winter et al. (1991) Nature 349:293-299; and U.S. Pat. No. 4,816,567); F(ab′)2 and F(ab) fragments; Fv molecules (noncovalent heterodimers, see, for example, Inbar et al. (1972) Proc. Natl. Acad. Sci. USA 69:2659-2662; and Ehrlich et al. (1980) Biochem. 19:4091-4096); single-chain Fv molecules (sFv) (see, e.g., Huston et al. (1988) Proc. Natl. Acad. Sci. USA 85:5879-5883); dimeric and trimeric antibody fragment constructs; minibodies (see, e.g., Pack et al. (1992) Biochem. 31:1579-1584; Cumber et al. (1992) J. Immunology 149B: 120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and any functional fragments obtained from such molecules, wherein such fragments retain specific-binding properties of the parent antibody molecule.

A “monoclonal antibody” is an antibody composition having a homogeneous antibody population. The term is not limited regarding the species or source of the antibody, nor is it intended to be limited by the manner in which it is made. The term encompasses whole immunoglobulins.

Methods of making polyclonal and monoclonal antibodies are known in the art. Polyclonal antibodies are generated by immunizing a suitable animal, such as a mouse, rat, rabbit, sheep, chicken, or goat, with an antigen of interest, such as a stem cell transformed with a gene encoding an antigen. In order to enhance immunogenicity, the antigen can be linked to a carrier prior to immunization. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Furthermore, the antigen may be conjugated to a bacterial toxoid, such as toxoid from diphtheria, tetanus, cholera, etc., in order to enhance the immunogenicity thereof.

Rabbits, sheep, and goats are preferred for the preparation of polyclonal sera when large volumes of sera are desired. These animals are good design choices also because of the availability of labeled anti-rabbit, anti-sheep, and anti-goat antibodies. Immunization is generally performed by mixing or emulsifying the antigen in saline or in an adjuvant such as Freund's complete adjuvant (FCA), and injecting the mixture or emulsion parenterally (generally subcutaneously or intramuscularly). The animal is generally boosted 2-6 weeks later with one or more injections of the antigen in saline, preferably using Freund's incomplete adjuvant (FIA). Antibodies may also be generated by in vitro immunization, using methods known in the art. Polyclonal antisera is then obtained from the immunized animal.

Monoclonal antibodies are generally prepared using the method of Kohler and Milstein, Nature (1975) 256:495-497, or a modification thereof. Typically, a mouse or rat is immunized as described above. However, rather than bleeding the animal to extract serum, the spleen (and optionally several large lymph nodes) is removed and dissociated into single cells. If desired, the spleen cells may be screened (after removal of non-specifically adherent cells) by applying a cell suspension toga plate or well coated with the antigen. B-cells, expressing membrane-bound immunoglobulin specific for the antigen, will bind to the plate, and are not rinsed away with the rest of the suspension. Resulting B-cells, or all dissociated spleen cells, are then induced to fuse with myeloma cells to form hybridomas, and are cultured in a selective medium (e.g., hypoxanthine, aminopterin, thymidine (HAT) medium). The resulting hybridomas are plated by limiting dilution, and are assayed for the production of antibodies which bind specifically to the immunizing antigen (and which do not bind to unrelated antigens). The selected monoclonal antibody-secreting hybridomas are then cultured either in vitro (e.g., in tissue culture bottles or hollow fiber reactors), or in vivo (e.g., as ascites in mice).

Monoclonal antibodies or portions thereof may be identified by first screening a B-cell cDNA library for DNA molecules that encode antibodies that specifically bind to the protein of interest, according to the method generally set forth by Huse et al. (1989) Science 246:1275-1281. The DNA molecule may then be cloned and amplified to obtain sequences that encode the antibody (or binding domain) of the desired specificity.

Human monoclonal antibodies are obtained by using human rather than murine hybridomas. See, e.g., Cote, et al. Monoclonal Antibodies and Cancer Therapy, Alan R. Liss, 1985, p. 77.

A “stem cell” is an undifferentiated pluripotent or multipotent cell with the ability to self-renew, to remain undifferentiated, and to become differentiated. Stem cells can divide without limit, at least for the lifetime of the animal in which they naturally reside. Stem cells are not terminally differentiated, i.e., they are not at the end of a pathway of differentiation. When a stem cell divides, each daughter cell can either remain a stem cell or it can embark on a course that leads to terminal differentiation. The stem cell can be an embryonic stem cell, a juvenile stem cell, or an adult stem cell, and can differentiate into neurons, myocytes, epithelial cells, blood cells, and other cells that may be at intermediate or terminal stages of differentiation. A “chimeric” stem cell is a stem cell with a portion of its DNA belonging to a heterologous organism.

An “embryonic stem cell” is a stem cell that is present in or isolated from an embryo. A “juvenile stem cell” is a stem cell that is present in or isolated from a juvenile. An “adult stem cell” is a stem cell that is present in or isolated from an adult. Either can be pluripotent, having the capacity to differentiate into each and every cell present in the organism, or multipotent, with the ability to differentiate into more than one cell type.

Embryonic stem cells, sometimes referred to as ES cells, derived from the inner cell mass of the embryo can act as pluripotent cells when placed into host blastocysts. Embryonic stem cells can be cultured and maintained in vitro while being kept in an undifferentiated state. Embryonic stem cells from mammals, including humans, mice, hamsters, and pigs, have been isolated and can be used in the invention. Embryonic stem cells can differentiate into any cell type in vivo and typically into a more limited variety of cells in vitro. Adult stem cells are more frequently multipotent than pluripotent; examples of multipotent adult stem cells include hematopoeitic stem cells, peripheral nervous system stem cells, central nervous system stem cells, and myogenic stem cells.

A “mesenchymal stem cell” (MSC) is an adult pluripotent stem cell progenitor, e.g., blast cell, of multiple mesenchymal lineages, including bone, cartilage, muscle, fat tissue, marrow stroma, and astrocytes. Mesenchyme is embryonic tissue of mesodermal origin, i.e., tissue that derives from the middle of three germ layers. The mesenchyme is populated by mesenchymal cells, which are typically stellate or fusiform in shape. The embryonic mesoderm gives rise to the musculoskeletal, blood, vascular, and urogenital systems, as well as connective tissue, i.e., the dermis. Mesenchymal stem cells can be found in bone marrow, blood, dermis, and periosteum. They can differentiate into, e.g., adipose, osseous, stroma, cartilaginous, elastic, and fibrous connective tissues. Their differentiation pathway, e.g., into cells such as osteoblasts and chondrocytes, depends on the agent(s) to which they are exposed.

A “hematopoeitic” cell is a cell involved in the process of hematopoeisis, i.e., the process of forming mature blood cells from precursor cells. In the adult, hematopoeisis takes place in the bone marrow. Earlier in development, hematopoeisis takes place at different sites during different stages of development; primitive blood cells arise in the yolk sac, and later, blood cells are formed in the liver, spleen, and bone marrow. Hematopoeisis undergoes complex regulation, including regulation by hormones, e.g., erythropoietin; growth factors, e.g., colony stimulating factors; and cytokines, e.g., interleukins.

Hematopoietic stem cells (HSCs) are formative pluripotential blast cells found in bone marrow and peripheral blood that are capable of differentiating into any of the specific types of hematopoietic or blood cells, such as erythrocytes, lymphocytes, macrophages, and megakaryocytes. The expression of a particular antigen or antigens on the cell surface or in the cytoplasm and the intensity of expression indicate the stage of maturation and lineage commitment of the hematopoietic stem cell. Hematopoietic stem cells can be obtained, for example, by subjecting low density mononuclear bone marrow cells to counterflow elutriation and then recovering CD34⁺ cells from the fractions containing the cells of smallest size. Thus, stem cells are isolated from bone marrow cells by panning the bone marrow cells with antibodies which bind unwanted cells, such as CD4+ and CD8+ (T cells), CD45+ (panB cells), GR-1 (granulocytes), and Iad (differentiated antigen presenting cells). The hematopoietic stem cells can be differentiated in vitro into clinically important immune cell types using cytokines such as, e.g., GM-CSF, IFN-γ and TNF-α. The above-described stem cells, as well as other stem cells, will find use in the present invention.

“Differentiation” is a progressive developmental change to a more specialized form or function. Cell differentiation is the process a cell undergoes as it matures to become an overtly specialized cell type. Differentiated cells have distinct characteristics, perform specific functions, and are less likely to divide than their less differentiated counterparts. An “undifferentiated” cell, e.g., an immature, embryonic, or primitive cell, typically has a non-specific appearance, may perform multiple, non-specific activities, and may perform poorly, if at all, in functions typically performed by differentiated cells.

“Dedifferentiation” is a process by which a mature cell returns to a less mature state. A “dedifferentiated cell” is one that has fewer characteristics of differentiation than it possesses at an earlier point in time. A “dedifferentiated state” is one in which a mature cell has returned or is returning to a less differentiated state, e.g., as in some cancers.

A “differentiation factor” is a factor that induces a cell to undergo a change in the direction of an overtly specialized cell type. An “anti-differentiation factor” is a factor that prevents or inhibits a cell from undergoing a change in the direction toward an overtly specialized cell type.

A “differentiated cell” is a cell that has developed from a relatively unspecialized phenotype to a more specialized phenotype, thereby attaining a particular degree of differentiation. For example, a progenitor cell type such as a hematopoietic stem cell can give rise to a more differentiated cell such as a monocyte or an erythrocyte. Differentiated cells can be isolated from embryonic or somatic cells using techniques known in the art.

A “blastocyst” is an embryo at an early stage of development in which the fertilized ovum has undergone cleavage, and a spherical layer of cells surrounding a fluid-filled cavity is forming, or has formed. The spherical layer of cells is the trophoectoderm. Inside the trophoectoderm is a cluster of cells termed the inner cell mass. The trophectoderm is the precursor of the placenta, and the inner cell mass is the precursor of the embryo. Cells of the early mammalian embryo are pluripotent.

A “transgenic mouse” has stably incorporated one or more genes from another cell or organism and can pass them on to successive generations.

A “knockout” mouse has a normal functional gene replaced by a non-functional form of the gene, with the function of that particular gene eliminated. Gene “knockout” produces model systems for studying inherited human diseases, investigating the nature of genetic diseases and the efficacy of different types of treatment, and for developing effective gene therapies to cure these diseases. For example, a “knockout” line of mutant mice homozygous for a null allele of the cystic fibrosis transmembrane regulator gene demonstrates symptoms similar to those of humans with cystic fibrosis. These mice provide a model system for studying this genetic disease and developing effective therapies.

A “SCID mouse” is a mouse model for severe combined immunodeficiency syndrome (SCID), which causes severe defects in the development of the immune system. These mice are deficient in, or completely lack, both T and B lymphocytes. The SCID mutation appears to impair the recombination of antigen receptor genes, causing a lack of functional T and B lymphocytes. Other hematopoeitic cell types appear to develop and function normally. SCID mice readily support normal lymphocyte differentiation and can be reconstituted with normal lymphocytes from syngeneic or allogeneic mice, or with human lymphocytes. These mice also support the growth of allogeneic and xenogeneic tumors. Therefore, SCID mice, which allow disseminated growth of a number of human tumors, particularly hematologic disorders and malignant melanoma, can be used to investigate malignancies.

A “non-obese diabetic” mouse is a mouse that serves as a model for type I diabetes. It is characterized by higher numbers of hyperactive pancreatic islet beta cells, high percentages of immature islets, elevated levels of some types of antigen-presenting cells and FasL+ cells, and abnormalities of extracellular matrix protein expression (Homo-Delarche (2001) Braz. J. Med. Biol. Res. 34:437-447).

An “Rb−/− mouse” is a mouse that provides a model for the study of retinoblastoma, a rare form of human childhood cancer that arises from neural precursor cells in the immature retina. The Rb gene is normally expressed in almost all cells of the body, and its product functions as a brake in the cell-division cycle. During the cell cycle, the Rb protein alternates between a phosphorylated and an unphosphorlyated state. Unphosphorylated Rb binds to regulatory proteins to prevent DNA replication. Loss of the Rb gene sets the cell cycle free from this restraint. Loss of Rb is a step in the progression of many cells toward malignancy. Its loss is sufficient to cause retinoblastoma, and also contributes to many of the more common cancers that arise by a more complex series of genetic changes, and appear later in life. Thus, although retinoblastoma is rare, cancers involving the Rb gene are not.

A “p53 −/− mouse” is a mouse that provides a model for the study of the tumor suppressor p53. Under normal conditions, p53 is present at low levels at most cell. Exposure to a stress, e.g., a mutagen such as ultraviolet light, raises the concentration of intracellular p53 and blocks cell proliferation, enabling cells to cope with DNA damage. Mice homozygous for a deletion in the p53 gene develop tumors at high frequency. In a variety of mouse models, absence of p53 facilitates tumorigenesis. Depending on the particular model system, loss of p53 either results in deregulated cell-cycle entry or aberrant apoptosis. The rapidity with which p53 null mice develop tumors makes them useful for evaluating agents for chemopreventative or therapeutic activities (Attardi and Jacks, (1999) Cell Mol. Life Sci. 55:48-63).

A “pseudo-pregnant” mouse is a female mouse that, although not pregnant, has a uterine environment that can support an implanted zygote. A pseudo-pregnant mouse can be produced by mating a female mouse with a vasectomized male. The stimulus of mating causes hormonal changes necessary to transform her uterus to a state capable of sustaining a pregnancy when a heterologous zygote is introduced into the lumen of the uterus or oviduct.

A “teratoma” is a malignant tumor that contains an undifferentiated stem cell population that has biochemical and developmental properties remarkably similar to those of the inner cell mass. Moreover, these stem cells not only divide, but can also differentiate into a wide variety of tissues, including gut and respiratory epithelia, muscle, nerve, cartilage, and bone. Once differentiated, these cells no longer divide, and are therefore no longer malignant. Such tumors can give rise to most of the tissue types in the body. Thus, the teratocarcinoma stem cells mimic early mammalian development, but the tumor they form is characterized by random, haphazard development.

A “pharmaceutically acceptable carrier,” “pharmaceutically acceptable diluent,” or “pharmaceutically acceptable excipient,” or “pharmaceutically acceptable vehicle,” used interchangeably herein, refer to a non-toxic solid, semisolid or liquid filler, diluent, encapsulating material or formulation auxiliary of any conventional type. A pharmaceutically acceptable carrier is non-toxic to recipients at the dosages and concentrations employed and is compatible with other ingredients of the formulation. For example, the carrier for a formulation containing polypeptides would not normally include oxidizing agents and other compounds that are known to be deleterious to polypeptides. Suitable carriers include, but are not limited to, water, dextrose, glycerol, saline, ethanol, and combinations thereof. The carrier can contain additional agents such as wetting or emulsifying agents, pH buffering agents, or adjuvants which enhance the effectiveness of the formulation. Adjuvants of the invention include, but are not limited to Freunds's, Montanide ISA Adjuvants [Seppic, Paris, France], Ribi's Adjuvants (Ribi ImmunoChem Research, Inc., Hamilton, Mont.), Hunter's TiterMax (CytRx Corp., Norcross, Ga.), Aluminum Salt Adjuvants (Alhydrogel—Superfos of Denmark/Accurate Chemical and Scientific Co., Westbury, N.Y.), Nitrocellulose-Adsorbed Protein, Encapsulated Antigens, and Gerbu Adjuvant (Gerbu Biotechnik GmbH, Gaiberg, Germany/C-C Biotech, Poway, Calif.). Topical carriers include liquid petroleum, isopropyl palmitate, polyethylene glycol, ethanol (95%), polyoxyethylene monolaurate (5%) in water, or sodium lauryl sulfate (5%) in water. Other materials such as anti-oxidants, humectants, viscosity stabilizers, and similar agents can be added as necessary. Percutaneous penetration enhancers such as Azone can also be included.

Various Aspects of the Invention

The invention provides methods and compositions for identifying functionally active secreted molecules and intracellular molecules, including those that are stimulatory and those that are inhibitory.

The invention provides methods and compositions for discovery of secreted and intracellular molecules that interact with each other to produce a biological function, such as a stimulatory function or an inhibitory function.

The invention provides methods and compositions to determine functions of biological molecules by providing phenotypic readouts for gain of function and loss of function.

The invention provides a cell library comprising a plurality of cells, where the plurality of cells is located in an addressable matrix.

The invention provides a cell library as above, where the matrix contains a plurality of spots or wells, each of which has an address, such as column 2, row 4, for example.

The invention provides a cell library as above, where the matrix contains a range of addressable spots, where the range is selected from any number from and in-between 5-50, 10-100, 20-200, 30-300, 40-400, 50-500, 60-600, 70-700, 80-800, 90-900, 100-1000, 250-2000, 350-3000, 450-4000, 550-5000, 650-6000, 750-7000, 850-8000, 950-9000, 1050-10000, and 10000-50000 or more.

The invention provides the matrix as above, with at least one cell, preferably more, being located in one or more spots or wells of the matrix.

The invention provides the cell library as above, where the matrix contains any suitable substrate or support, such as a 96-well plate, a 384-well or larger plate, a glass slide containing depressions or wells in rows and columns, and such similar substrates that are suitable for high throughput analysis, or can be adapted for a robotics system.

The invention provides a cell library as above, where each address in the matrix contains either the same or different number of cells, or the same or different type of cells.

The invention provides a library of cells as above, where the cells are transformed cells, and the transformed cells are manipulated to contain introduced nucleic acid molecules that encode secreted molecules, transmembrane molecules, or intracellular molecules, where such molecules have either a stimulatory or inhibitory effect on the transformed cells.

The invention provides a library of transformed cells, as above, where the transformed cells are selected from stem cells, T cells, B cells, pancreatic islet cells, COS cells and other cells that are naturally capable of secreting proteins or expressing transmembrane proteins.

The invention provides a library of cells as above, where the cells are readout cells, also referred to as reporter cells, and the readout cells are capable of exhibiting an observable biological effect or phenotype when placed in contact with a biological molecule that has either stimulatory or inhibitory function.

The invention provides a library of cells as above, where the cells are transformed cells, and the transformed cells are manipulated to contain introduced nucleic acid molecules that encode secreted molecules, transmembrane molecules, or intracellular molecules, where such molecules have either a stimulatory or inhibitory effect on the transformed cells.

The invention provides a library of transformed cells as above, where the transformed cells exhibit a gain of function, for example, the cells acquire the ability to secrete certain proteins.

The invention provides a library of transformed cells showing gain of function, as above, where such cells are further transformed by additional introduced nucleic acid molecules that affect such gain of function, for example, by introducing nucleic acid molecules that knock out function.

The invention provides the twice-transformed library of cells as above, where the nucleic acid molecules that knock out function are inhibitory molecules, such as RNAi molecules.

The invention provides a library of readout cells as above, where the readout cells are selected from stem cells, T cells, B cells, CNS cells, cartilage cells, bone cells, pancreatic islet cells, fat cells, oocytes, and eggs.

The invention provides a library of stem cells as readout cells, where the stem cells differentiate to produce cells selected from CNS cells including brain cells, neurons, astrocytes, glial cells, T cells, B cells, cartilage cells, bone cells, pancreatic islet cells, fat cells, heart cells, liver cells, kidney cells, lung cells, muscle cells, and eye cells. The stem cells can be derived from any mammalian organism, such as, for example, human or mouse. The readout cells can be derived from other animals, such as, for example, frogs, rabbits, cows, pigs, and the like.

The invention provides a library of readout cells as above, where the readout cells are stem cells, and the stem cells are embryonic or adult stem cells.

The invention provides a library of stem cells as above, where the stem cells are adult stem cells, and the adult stem cells are isolated from a tissue selected from bone marrow, brain, thymus, liver, kidney, spleen, placenta, lung and other tissues in the body.

The invention provides a stem cell library as above, where at least some of the stem cells are transformed with at least one, and optionally 2, or 3, or 4, or 5, or more introduced nucleic acid molecules.

The invention provides a stem cell library, as above, where the stem cells are used as readouts to determine the function and/or effect of nucleic acid molecules introduced into such stem cells. For example, the introduced nucleic acid molecules may encode a factor that causes the stem cells to differentiate into cells of different lineages.

The invention provides a library of stem cells as above, where the stem cells are differentiated into cells of different lineages, including but not limited to: cardiomyocytes, T cells, B cells, leukocytes, other cells of the hematopoietic system, neurons, astrocytes, glia cells, other cells of the CNS, liver cells, bone cells, cartilage cells, pancreatic islet cells, kidney cells, muscle cells, and other cells of the body.

The invention provides a stem cell library as above, where the stem cells are placed in contact with proteins or fragments of protein having biological activity, for example, to use the stem cells as readouts to determine the function and/or effect of one or more proteins, or protein fragments on the growth and/or differentiation of such stem cells.

The invention provides a library of stem cells as above, where the added proteins or fragments are molecules present in the medium in which the stem cells are suspended.

The invention provides a library of stem cells, as above, where the stem cells are in contact with proteins or fragments, and the proteins or fragments are present in the form of other cells expressing such proteins or fragments.

The invention provides a library of stem cells in contact with proteins and fragments, as above, where the proteins and fragments are expressed on the cell surface of other cells or are secreted by the other cells.

The invention provides a library of stem cells as above that contains introduced nucleic acid molecules, and the introduced nucleic acid molecules encode intracellular molecules, the intracellular molecules being selected from transcription factors, nuclear receptors, kinases, phosphatases, proteases, and ion channels.

The invention provides a combined library of transformed cells and readout cells that are in physical contact with each other. Such a combined library can be used, for example, to study molecular interaction.

The invention provides a combined library as above, where the transformed cells express a protein or active fragment, such as in the form of a ligand, and a nucleic acid molecule introduced into the readout stem cells encodes a receptor.

The invention provides a library of a transformed cells, as above, where the transformed cells are selected from COS cells, T cells, B cells, pancreatic islet cells and the like, of any species origin, including human, mouse, bird, fish, worm, insect, and yeast.

The invention provides a stem cell library as above, where the stem cells are transformed with introduced nucleic acid molecules, and the introduced nucleic acid molecules are of any origin, including mammalian or non-mammalian origin, such as mouse, human, chicken, fish, flies, or others.

The invention provides a library of stem cells as above, where the stem cells are the source of secreted molecules and are placed in contact with other stems cells to determine the effect of the secreted molecules on the growth and/or differentiation of the other stem cells.

The invention provides a stem cell library comprising a plurality of stem cells, where the plurality of stem cells is situated in a matrix, and comprises a first stem cell that is transformed with a first nucleic acid molecule that encodes a first protein, a second stem cell that is transformed with a second nucleic acid molecule that encodes a second protein, a third stem cell that is transformed with a third nucleic acid molecule that encodes a third protein, a fourth stem cell that is transformed with a fourth nucleic acid molecule that encodes a fourth protein, and so on, up to any tens, hundreds, or thousands, or tens of thousands of stem cells, each transformed with a different nucleic acid molecule encoding a different protein.

The invention provides a stem cell library as above, containing a first stem cell that is transformed with a first nucleic acid molecule that encodes a first protein, and at least a second stem cell that is transformed with at least one different nucleic acid molecule encoding at least one different protein.

The invention provides a stem cell library as above, where the stem cells are transformed with nucleic acid molecules encoding proteins of the same family or proteins of different families, selected from secreted proteins, transmembrane proteins, kinases, phosphatases, proteases, transcription factors, ion channels, kinesins, defensins, and others.

The invention provides a collection of stem cells located at N different addresses, where N is a positive integer, selected from the ranges of 5-100, 10-200, 15-300, 20-400, 30-500, 40-600, 50-700 or more; and optionally, 90-800, 900-1000, 1000-2000, 2000-3000, 3000-4000, 4000-5000, 5000-6000, 6000-7000 or more; further optionally, 8000-10000, 10000-20000 or more.

The invention provides the library of stem cells as above, where the stem cells are incubated with (a) one different stem cell transformed with a first different nucleic acid molecule; (b) two different stem cells: one transformed with a first different nucleic acid molecule and the other transformed with second different nucleic acid molecule, respectively; (c) three different stem cells: one transformed with a first different nucleic acid molecule, a second transformed with a second different nucleic acid molecule, and a third transformed with a third different nucleic acid molecule, respectively; (d) four different stem cells: one transformed with a first different nucleic acid molecule, a second transformed with a second different nucleic acid molecule, a third transformed with a third different nucleic acid molecule, and a fourth transformed with a fourth different nucleic acid molecule, respectively; and/or five different stem cells: one transformed with a first different nucleic acid molecule, a second transformed with a second different nucleic acid molecule, a third transformed with a third different nucleic acid molecule, a fourth transformed with a fourth different nucleic acid molecule, and a fifth transformed with a fifth different nucleic acid molecule, respectively.

The invention provides a library of stem cells as above, where the stem cells are transformed with introduced DNA or RNA, with or without the use of any accompanying facilitating agents, such as lipofectamine, for example.

The invention provides a library of transformed stem cells as above, where the stem cells are transformed with introduced DNA or RNA, and the DNA or RNA encodes proteins or families of proteins selected from secreted proteins, transmembrane proteins, kinases, phosphatases, proteases, ion channels, transcription factors, kinesins, defensins, and others. Such secreted proteins include but are not limited to growth factors, cytokines, lymphokines, chemokines, interleukins, interferons, and extracellular portions of transmembrane proteins that may be cleaved from a cell.

The invention provides a library of transformed stem cells as above, where the stem cells secrete proteins such as growth factors, and the proteins and growth factors fall within a family selected from epidermal growth factor (“EGF”), fibroblast growth factor (“FGF), keratinocyte growth factor (“KGF”), platelet-derived growth factor (“PDGF”), insulin-like growth factor (“IGF”), nerve growth factor “NGF”), brain derived growth factor (“BDGF”), hepatocyte growth factor (“HGF”), transforming growth factor (“TGF”)for example, TGF-β, and bone morphogenic protein (“BMP”), for example BMP4, granulocyte-macrophage colony stimulating factor (“GM-CSF”), macrophage colony stimulating factor (“M-CSF”), colony stimulating factor (“CSF”), stem cell growth factor (“SGF”), erythropoietin (“EPO”), transforming growth factor (“TGF”), for example, TFG-β, and CD40L (a ligand). The secreted proteins also include interleukins, such as IL-1, IL-2, IL-3, IL-4, IL-5, IL-6 and IL-12, and MCP-1, CCL5, and interferons, such as IFN-α, IFN-β, and IFN-γ. The proteins herein are human proteins or non-human animal proteins, including mouse proteins, rat proteins, chicken proteins, and fish proteins.

The invention provides a method of identifying secreted molecules, including the steps of transforming a library of cells with nucleic acid molecules suspected of encoding secreted molecules, and incubating such transformed cells with readout cells.

The invention provides a method of identifying secreted molecules as above, where the transformed cells and readout cells are, respectively, selected from stem cells, differentiated stem cells, T cells, B cells, pancreatic islet cells, COS cells, cancer cells, hepatocytes, liver cells, lung cells, bone marrow cells, and neuronal cells of any species origin, including human, mouse, or other mammals.

The invention provides a stem cell library as above, where the stem cells, with or without the addition of introduced nucleic acid molecules, are allowed to proliferate and/or differentiate.

The invention provides a stem cell library, as above, where the stem cells are manipulated to contain nucleic acid molecules each comprising a promoter. The promoter is selected from an inducible promoter, a conditionally active promoter (for example, cre-lox system), a constitutive promoter, and a tissue-specific promoter.

The invention provides the library of transformed stem cells, as above, where the stem cells are mouse ES cells, and the introduced nucleic acid molecules are targeted to the ROSA 26 locus of the stem cells.

The invention provides a method of determining the function of a first protein encoded by a first nucleic acid molecule, where the method comprises allowing a first transformed stem cell to grow, where the first transformed stem cell is transformed with a first nucleic acid molecule that is targeted to a first locus; and observing the growth, differentiation, inhibition of growth or inhibition of differentiation of the first transformed stem cell to determine a function of the first protein.

The invention provides a method of determining function of a library of proteins, where the method comprises the steps of transforming a stem cell library as above with a plurality of nucleic acid molecules that encode a plurality of proteins or biologically active fragments thereof, allowing the stem cells in the library to grow or differentiate; and observing growth, differentiation, or inhibition of growth or differentiation of the stem cells in the library.

The invention provides a method of massively parallel screening for protein activity, comprising: providing a combinatorial library of stem cells, wherein the library comprises a plurality of stem cells in an addressable matrix, where stem cells at each address of the matrix are transformed with a plurality of distinguishable nucleic acid molecules encoding a plurality of proteins; and monitoring the library of stem cells for growth, differentiation, or inhibition thereof.

The invention provides a library of cells, other than stem cells, that provide readouts for biological activities or functions. Such a library includes, but is not limited to, a library of any medically relevant cell types such as T cells or B cells, activated or non-activated; cancer cells, whether primary cancer cells or from cancer cell lines; virus-infected cells; parasite-infected cells, fungus-infected cells; bacteria-infected cells; prion-infected cells; and cells from diseased tissues. The library of non-stem cells can be used to study gene or protein function; for identifying novel secreted factors; for identifying novel protein function, etc. by monitoring growth, normal or abnormal, or inhibition of growth or biological activity of the cells in the library.

The invention provides an embryonic stem cell comprising at least one introduced nucleic acid molecule, encoding a protein of interest. In certain embodiments, the introduced nucleic acid molecule encodes at least one transmembrane protein or active fragments thereof, and the embryonic stem cell expresses at least one transmembrane protein or active fragments on its cell surface. The embryonic stem cell can be an animal cell and may be selected from a mouse cell, a rat cell, a guinea pig cell, a sheep cell, a goat cell, a bovine cell, a rabbit cell, a canine cell, a feline cell, a porcine cell, and an ovine cell. The at least one transmembrane protein may be, but need not be, substantially identical to an animal transmembrane protein, such as, but not limited to, a human transmembrane protein. The embryonic stem cell may also comprise a plurality of nucleic acid molecules, wherein the plurality of nucleic acid molecules encode a plurality of transmembrane proteins or active fragments thereof.

The invention provides a composition comprising embryonic stem cells that comprise introduced nucleic acid molecules, wherein the introduced nucleic acid molecules encode at least one protein, such as a transmembrane protein, or active fragments thereof, and a pharmaceutically acceptable carrier. The embryonic stem cells may comprise a plurality of introduced nucleic acid molecules, and the plurality of nucleic acid molecules may encode a plurality of proteins, such as transmembrane proteins, or active fragments thereof. In certain embodiments, the composition can be used as an immunogen. Alternatively, fragments of the stem cells described above, such as membrane fragments including a protein such as a transmembrane protein, can be used as an immunogen.

The invention provides a method of producing antibodies to at least one protein or active fragments thereof, such as a transmembrane protein or active fragments thereof, comprising the steps of providing an embryonic stem cell that comprises at least one introduced nucleic acid molecule, wherein at least one introduced nucleic acid molecule encodes at least one protein, such as a transmembrane protein or active fragments thereof, and wherein the embryonic stem cell expresses at least one protein or active fragments thereof on its cell surface; immunizing a host with the embryonic stem cell; and recovering antibodies specific to at least one protein or active fragments thereof from serum of the host. In certain embodiments of the above method, the embryonic stem cell is derived from a species that is other than the host species or is derived from a species that is the same as the host species. The protein can be derived from a species that is other than the embryonic cell species.

The invention provides a method of producing antibodies to at least one protein or active fragments thereof, such as a transmembrane protein or active fragments thereof, comprising the steps of obtaining a nucleic acid encoding the protein or active fragments thereof, and introducing it to the stem cell, wherein the introduced nucleic acid molecule encodes at least one protein, such as a transmembrane protein or active fragments thereof, and wherein the embryonic stem cell expresses the protein or active fragments thereof on its cell surface, and the fragment comprises the protein, such as a membrane fragment that includes a transmembrane protein; immunizing a host with the embryonic stem cell fragment; and recovering antibodies specific to at least one protein or active fragments thereof from serum of the host. In certain embodiments of the above method, the embryonic stem cell is derived from a species that is other than the host species or is derived from a species that is the same as the host species. The at least one protein can be derived from a species that is other than the embryonic cell species.

The invention provides a method of producing antibodies to at least one protein, such as a transmembrane protein, or active fragments thereof comprising the steps of providing an embryonic stem cell that comprises at least one introduced nucleic acid molecule, wherein at least one introduced nucleic acid molecule encodes at least one protein or active fragments thereof, and wherein the embryonic stem cell expresses at least one protein or active fragment thereof on its cell surface; immunizing a host with the embryonic stem cell; and recovering antibodies specific to at least one protein or active fragment thereof from spleen cells of the host. In certain embodiments of the above method, the embryonic stem cell is derived from a species that is other than the host species or is derived from a species that is the same as the host species. The at least one protein can be derived from a species that is other than the embryonic cell species. The method can further comprise the step of fusing a spleen cell that produces an antibody specific to at least one protein or active fragment thereof with an immortalized cell to produce a hybridoma. In additional embodiments of the above method, the method further comprises the step of extracting mRNA molecules from the spleen cells and selecting one or more mRNA molecules that encode one or more antibodies specific to at least one or more proteins. The mRNA molecules can be used to make a cDNA library. Additionally, the mRNA molecules can be expressed in an in vitro cell free translation process to produce an antibody or fragments thereof in vitro. Additional embodiments of the above method further comprise the step of selecting one or more cDNA molecules that encode one or more antibodies specific to at least one or more proteins. The cDNA molecule can be expressed in an expression system to express an antibody or active fragments thereof.

The invention provides a method of producing antibodies to at least one protein, such as a transmembrane protein, or active fragments thereof comprising the steps of providing a fragment of an embryonic stem cell that comprises at least one introduced nucleic acid molecule, wherein at least one introduced nucleic acid molecule encodes at least one protein, such as a transmembrane protein or active fragments thereof, and wherein the embryonic stem cell expresses at least one protein or active fragments on its cell surface, and the fragment comprises the protein, such as membrane fragment that includes a transmembrane protein; immunizing a host with the embryonic stem cell; and recovering antibodies specific to at least one protein or active fragments from spleen cells of the host. In certain embodiments of the above method, the embryonic stem cell is derived from a species that is other than the host species or is derived from a species that is the same as the host species. The at least one protein can be derived from a species that is other than the embryonic cell species. The method can further comprise the step of fusing a spleen cell that produces an antibody specific to at least one protein with an immortalized cell to produce a hybridoma. In additional embodiments of the above method, the method further comprises the step of extracting mRNA molecules from the spleen cells and selecting one or more mRNA molecules that encode one or more antibodies specific to at least one or more proteins. The mRNA molecules can be used to make a cDNA library. Additionally, the mRNA molecules can be expressed in an in vitro cell free translation process to produce an antibody or fragments thereof in vitro. Additional embodiments of the above method further comprise the step of selecting one or more cDNA molecules that encode one or more antibodies specific to at least one or more proteins. The cDNA molecule can be expressed in an expression system to express an antibody of active fragments thereof.

The invention provides an antibody produced by any of the methods above.

The invention provides a method of producing a protein or an active fragment thereof comprising the steps of providing an embryonic stem cell that comprises at least one introduced nucleic acid molecule, wherein at least one introduced nucleic acid molecule encodes at least one protein or an active fragment thereof, wherein at least one protein is a heterologous protein and wherein the embryonic stem cell expresses the heterologous protein; and recovering the heterologous protein from the embryonic stem cell. The method may further comprise the step of substantially purifying the heterologous protein.

The invention provides a protein or an active fragment thereof produced by the method above.

The invention provides a method of determining gene function comprising targeting a gene of interest to a particular locus of a stem cell, such as the ROSA 26 locus of a mouse embryonic stem cell, culturing the embryonic stem cell under conditions that provide for differentiation of the embryonic stem cell into a differentiated cell, including, but not limited to cardiomyocytes, T cells, B cells, leukocytes, other cells of the hematopoietic system, neurons, astrocytes, glia cells, other cells of the CNS, liver cells, bone cells, cartilage cells, pancreatic islet cells, kidney cells, muscle cells, and other cells of the body, expressing the protein encoded by the gene of interest and determining the effect of the protein on the differentiated cell.

The invention provides a method of determining gene function in vivo comprising providing an embryonic stem cell, such as a mouse embryonic stem cell, targeting a gene of interest to a particular locus of the embryonic stem cell, such as the ROSA 26 locus of a mouse embryonic stem cell, providing the transformed embryonic stem cell to a blastocyst, implanting the blastocyst into an animal, such as a non-human animal, e.g., a mouse, allowing the blastocyst to develop into an embryo, fetus or an animal, in vivo, to produce a chimeric embryo, chimeric fetus, or chimeric animal, such as a non-human animal, e.g., a mouse, wherein the embryo, fetus or non-human animal produces the product encoded by the gene of interest in multiple tissues, and determining the effect of the gene product on the embryo, fetus or animal.

The invention provides a cell line produced from cells or tissues obtained from the chimeric embryo, fetus or animal above.

The invention provides a method of determining gene function in vivo comprising providing an embryonic stem cell, such as a mouse embryonic stem cell, targeting a gene of interest to a particular locus of the embryonic stem cell, such as the ROSA 26 locus of a mouse embryonic stem cell, providing the transformed embryonic stem cell to a tissue of an animal, for example implanting the embryonic stem cell in a nude mouse, and allowing the embryonic stem cell to develop into a chimeric neoplasm, such as a teratoma, and determining the effect of the gene product on the neoplasm.

The invention provides a cell line developed from the chimeric neoplasm above.

The invention provides a modified stem cell comprising a plurality of chromosomes and at least a first heterologous nucleic acid molecule, wherein the modified stem cell can differentiate into a plurality of cell types; the first heterologous nucleic acid molecule is integrated into a chromosome of the modified stem cell at a first locus, whereby upon differentiation of the modified stem cell, the first heterologous nucleic acid is expressed into each of the cell types; wherein the first heterologous nucleic acid molecule encodes a first polypeptide selected from secreted proteins, extracellular domains of transmembrane proteins, and active fragments thereof; and wherein the first polypeptide is other than beta-galactosidase and a recombinase.

The invention provides a modified stem cell comprising a plurality of chromosomes and at least a first heterologous nucleic acid molecule, wherein the modified stem cell can differentiate into a plurality of cell types; the first heterologous nucleic acid molecule is integrated into a chromosome of the modified stem cell at a first locus, whereby upon differentiation of the modified stem cell, the first heterologous nucleic acid is expressed in the plurality of differentiated cell types; wherein the first heterologous nucleic acid molecule encodes a first polypeptide selected from single transmembrane proteins, multi-transmembrane proteins, kinases, proteases, phosphatases, phosphodiesterases, kinesins, histone deacetylases, hormone receptors, ubiquitin E3 ligases, and active fragments thereof; and wherein the first polypeptide is other than beta-galactosidase and a recombinase.

The invention provides a modified stem cell comprising a plurality of chromosomes and at least a first heterologous nucleic acid molecule, wherein the modified stem cell can differentiate into a plurality of cell types; the first heterologous nucleic acid molecule is integrated into a chromosome of the modified stem cell at a first locus, whereby upon differentiation of the modified stem cell, the first heterologous nucleic acid is expressed in the plurality of differentiated cell types; wherein the first heterologous nucleic acid molecule encodes a first polypeptide that is an episomal plasmid maintenance molecule or an active fragment thereof, and wherein the first polypeptide is other than beta-galactosidase and a recombinase.

The invention provides a modified blastocyst from a first animal that comprises a modified stem cell from a second animal, wherein the modified stem cell comprises a stem cell that comprises a plurality of chromosomes and at least a first heterologous nucleic acid molecule, wherein the modified stem cell can differentiate into a plurality of cell types; the first heterologous nucleic acid molecule is integrated into a chromosome of the modified stem cell at a first locus, whereby upon differentiation of the modified stem cell, the first heterologous nucleic acid is expressed in the plurality of differentiated cell types; wherein the first heterologous nucleic acid molecule encodes a first polypeptide selected from secreted proteins, extracellular domains of transmembrane proteins, and active fragments thereof; and wherein the first polypeptide is other than beta-galactosidase and a recombinase.

The invention provides a modified blastocyst comprising a blastocyst from a first animal that comprises a modified stem cell from a second animal, wherein the modified stem cell comprises a stem cell that comprises a plurality of chromosomes and at least a first heterologous nucleic acid molecule, wherein the modified stem cell can differentiate into a plurality of cell types; the first heterologous nucleic acid molecule is integrated into a chromosome of the modified stem cell at a first locus, whereby upon differentiation of the modified stem cell, the first heterologous nucleic acid is expressed in the plurality of differentiated cell types; wherein the first heterologous nucleic acid molecule encodes a first polypeptide selected from single transmembrane proteins, multi-transmembrane proteins, kinases, proteases, phosphatases, phosphodiesterases, kinesins, histone deacetylases, hormone receptors, and ubiquitin E3 ligases, and active fragments thereof; and wherein the first polypeptide is other than beta-galactosidase and a recombinase.

The invention provides a modified blastocyst comprising a blastocyst from a first animal that comprises a modified stem cell from a second animal, wherein the modified stem cell comprises a stem cell that comprises a plurality of chromosomes and at least a first heterologous nucleic acid molecule, wherein the modified stem cell can differentiate into a plurality of cell types; the first heterologous nucleic acid molecule is integrated into a chromosome of the modified stem cell at a first locus, whereby upon differentiation of the modified stem cell, the first heterologous nucleic acid is expressed in the plurality of differentiated cell types; wherein the first heterologous nucleic acid molecule encodes a first polypeptide that is an episomal plasmid maintenance molecule or an active fragment thereof; and wherein the first polypeptide is other than beta-galactosidase and a recombinase.

The invention provides a non-human chimeric animal developed from a modified blastocyst comprising a blastocyst from a first animal that comprises a modified stem cell from a second animal or a progeny thereof, wherein the modified stem cell comprises a stem cell that comprises a plurality of chromosomes and at least a first heterologous nucleic acid molecule, wherein the modified stem cell can differentiate into a plurality of cell types; the first heterologous nucleic acid molecule is integrated into a chromosome of the modified stem cell at a first locus, whereby upon differentiation of the modified stem cell, the first heterologous nucleic acid is expressed in the plurality of differentiated cell types; wherein the first heterologous nucleic acid molecule encodes a first polypeptide selected from secreted proteins, extracellular domains of transmembrane proteins, and active fragments thereof; and wherein the first polypeptide is other than beta-galactosidase and a recombinase.

The invention provides a non-human chimeric animal developed from a modified blastocyst comprising a blastocyst from a first animal that comprises a modified stem cell from a second animal or a progeny thereof, wherein the modified stem cell comprises a plurality of chromosomes and at least a first heterologous nucleic acid molecule, wherein the modified stem cell can differentiate into a plurality of cell types; the first heterologous nucleic acid molecule is integrated into a chromosome of the modified stem cell at a first locus, whereby upon differentiation of the modified stem cell, the first heterologous nucleic acid is expressed in the plurality of differentiated cell types; wherein the first heterologous nucleic acid molecule encodes a first polypeptide selected from single transmembrane proteins, multi-transmembrane proteins, kinases, proteases, phosphatases, phosphodiesterases, kinesins, histone deacetylases, hormone receptors, ubiquitin E3 ligases and active fragments thereof; and wherein the first polypeptide is other than beta-galactosidase and a recombinase.

The invention provides a non-human chimeric animal developed from a modified blastocyst comprising a blastocyst from a first animal that comprises a modified stem cell from a second animal or a progeny thereof, wherein the modified stem cell comprises a stem cell that comprises a plurality of chromosomes and at least a first heterologous nucleic acid molecule; wherein the modified stem cell can differentiate into a plurality of cell types; the first heterologous nucleic acid molecule is integrated into a chromosome of the modified stem cell at a first locus, whereby upon differentiation of the modified stem cell, the first heterologous nucleic acid is expressed in the plurality of differentiated cell types; wherein the first heterologous nucleic acid molecule encodes a first polypeptide that is an episomal plasmid maintenance molecule or an active fragment thereof; and wherein the first polypeptide is other than beta-galactosidase and a recombinase.

The invention provides a method of making a modified blastocyst, comprising the steps of obtaining a blastocyst from a first animal; obtaining a modified stem cell that comprises a plurality of chromosomes and at least a first heterologous nucleic acid molecule, wherein the modified stem cell can differentiate into a plurality of cell types; the first heterologous nucleic acid molecule is integrated into a chromosome of the modified stem cell at a first locus, whereby upon differentiation of the modified stem cell, the first heterologous nucleic acid is expressed in the plurality of differentiated cell types; wherein the first heterologous nucleic acid molecule encodes a first polypeptide that is an a episomal plasmid maintenance molecule or an active fragment thereof; and wherein the first polypeptide is other than beta-galactosidase and a recombinase; and introducing the modified stem cell into the blastocyst to produce the modified blastocyst.

The invention provides a method of making a non-human chimeric animal comprising the steps of obtaining a modified blastocyst; implanting the modified blastocyst into a pseudo-pregnant animal; and allowing the blastocyst to develop into a non-human chimeric animal; wherein the modified blastocyst comprises a blastocyst from a first animal that comprises modified stem cell from a second animal, wherein the modified stem cell comprises a stem cell that comprises a plurality of chromosomes and at least a first heterologous nucleic acid molecule, wherein the modified stem cell can differentiate into a plurality of cell types; the first heterologous nucleic acid molecule is integrated into a chromosome of the modified stem cell at a first locus, whereby upon differentiation of the modified stem cell, the first heterologous nucleic acid is expressed in the plurality of differentiated cell types; wherein the first heterologous nucleic acid molecule encodes a first polypeptide selected from secreted proteins, extracellular domains of transmembrane proteins, and active fragments thereof; and wherein the first polypeptide is other than beta-galactosidase and a recombinase.

The invention provides a method of making a non-human chimeric animal comprising the steps of obtaining a modified blastocyst; implanting the modified blastocyst into a pseudo pregnant non-human animal; and allowing the blastocyst to develop into a non-human chimeric animal; wherein the modified blastocyst comprises a blastocyst from a first animal that comprises one or more modified stem cells from a second animal; wherein the modified stem cell comprises a stem cell that comprises a plurality of chromosomes and at least a first heterologous nucleic acid molecule; wherein the modified stem cell can differentiate into a plurality of cell types; the first heterologous nucleic acid molecule is integrated into a chromosome of the modified stem cell at a first locus, whereby upon differentiation of the modified stem cell, the first heterologous nucleic acid is expressed in the plurality of differentiated cell types; wherein the first heterologous nucleic acid molecule encodes a first polypeptide selected from single transmembrane proteins, multi-transmembrane proteins, kinases, proteases, phosphatases, phosphodiesterases, kinesins, histone deacetylases, hormone receptors, ubiquitin E3 ligases and active fragments thereof; and wherein the first polypeptide is other than beta-galactosidase and a recombinase.

The invention provides a method of making a non-human chimeric animal comprising the steps of obtaining a modified blastocyst implanting the modified blastocyst into a pseudo-pregnant non-human animal; and allowing the blastocyst to develop into a non-human chimeric animal, wherein the modified blastocyst comprises a blastocyst from a first animal that comprises one or more modified stem cells from a second animal, wherein the modified stem cell comprises a plurality of chromosomes and at least a first heterologous nucleic acid molecule, wherein the modified stem cell can differentiate into a plurality of cell types; the first heterologous nucleic acid molecule is integrated into a chromosome of the modified stem cell at a first locus, whereby upon differentiation of the modified stem cell, the first heterologous nucleic acid is expressed in the plurality of differentiated cell types; wherein the first heterologous nucleic acid molecule encodes a first polypeptide that is an episomal plasmid maintenance molecule or an active fragment thereof; and wherein the first polypeptide is other than beta-galactosidase and a recombinase.

The invention provides a composition comprising a first modified and at least a second modified stem cell, wherein the first modified stem cell comprises at least a first heterologous nucleic acid molecule that encodes a first polypeptide, and the second modified stem cell comprises at least a second heterologous nucleic acid molecule that encodes a second polypeptide; wherein the first polypeptide encodes a secreted factor and the second polypeptide encodes a receptor; wherein the first nucleic acid integrates at a first locus of a chromosome of the first modified stem cell and the second nucleic acid integrates at a second locus of a chromosome of the second modified stem cell; and wherein the first and second locus are identical.

The invention provides a composition comprising a first library of modified stem cells and a second library of modified stem cells, wherein the first library of modified stem cells comprises a plurality of modified stem cells, wherein the plurality of modified stem cells comprises at least a first modified stem cell that is transfected with a first heterologous nucleic acid molecule that encodes a first member of a first family of proteins or an active fragment thereof and at least a second modified stem cell that is transfected with a second heterologous nucleic acid molecule that encodes a second member of the first family of proteins or an active fragment thereof, wherein the second library of modified stem cells comprises a plurality of modified stem cells, wherein the plurality of modified stem cells comprises at least a first modified stem cell that is transfected with a first heterologous nucleic acid molecule that encodes a first member of a second family of proteins or an active fragment thereof and at least a second modified stem cell that is transfected with a second heterologous nucleic acid molecule that encodes a second member of the second family of proteins or an active fragment thereof; and wherein the first family of proteins is a family of secreted proteins or extracellular domains of single transmembrane proteins, and the second family of proteins is a family of receptors.

The invention provides a modified mesenchymal stem cell comprising at least one first heterologous nucleic acid sequence encoding at least one first therapeutic molecule for a disease, disorder, syndrome, or condition, wherein said sequence is other than an anti-cancer agent.

The invention provides a modified mesenchymal stem cell comprising a mesenchymal stem cell that comprises at least one first heterologous nucleic acid sequence, wherein the first heterologous nucleic acid sequence encodes a therapeutic factor that is therapeutic for cancer and is other than a cytokine, a hormone, an extracellular matrix component, an enzyme, a signaling molecule, an anti-angiogenic polypeptide, an oncolytic virus, interferon-α, or interferon-β.

The invention provides a chimeric non-human animal stem cell comprising a non-human animal stem cell and at least one first heterologous nucleic acid sequence, wherein the first heterologous nucleic acid sequence encodes a first human polypeptide other than β-galactosidase, wherein the first heterologous nucleic acid sequence is inserted at a first locus of a chromosome of the non-human animal, and wherein insertion of the first heterologous nucleic acid sequence at the first locus enables expression of the polypeptide in the chimeric stem cell in both a differentiated and undifferentiated state.

The invention provides a chimeric non-human blastocyst comprising at least one chimeric non-human animal stem cell, wherein the chimeric non-human animal stem cell comprises a non-human animal stem cell and at least one first heterologous nucleic acid sequence, wherein the first heterologous nucleic acid sequence encodes a first human polypeptide; wherein the first heterologous nucleic acid sequence is inserted at a first locus of a chromosome of the non-human animal stem cell; and wherein insertion of the first heterologous nucleic acid sequence at the first locust enables expression of the polypeptide in the chimeric stem cell.

The invention provides methods and compositions for identifying functionally active biological molecules in a high throughput manner.

These and other aspects of the present invention will become evident upon reference to the following detailed description and attached drawings. In addition, various references are set forth herein which describe in more detail certain procedures or compositions, and are therefore incorporated by reference in their entirety.

Modes of Carrying Out the Invention

In one aspect of the invention, stem cells are transformed with nucleic acids encoding a protein or a fragment of a protein. The stem cell can be an embryonic stem cell, and the protein can be a secreted protein or an extracellular domain of a transmembrane protein. Stem cells can be transformed with nucleic acids encoding different proteins thereby creating a library of stem cells. The library of transformed stem cells can be allowed to differentiate to form different types of cells, and the activities of the proteins can be assayed by their effects on stem cell differentiation.

Thus, in one aspect of the invention, the stem cells in different wells can contain stem cells from the same source but that have been transformed with different nucleic acid molecules (hereafter, “Library Configuration 1”). In this Library Configuration 1, the effect of the nucleic acid molecules, and molecules encoded thereby, on the growth and differentiation of the transformed stem cells can be observed, detected or identified. In addition, such a library of transformed stem cells can act as a library of secreted molecules, to the extent the transforming nucleic acid molecules encode secreted molecules. The library of secreted molecules can be placed in contact with another set of cells, readout cells, to determine the effect of the secreted molecules on the readout cells. In another aspect of the invention, the library of transformed stem cells can be allowed to differentiate in the presence of an external stimulus. The external stimulus can be, for example, a specific factor that promotes the differentiation of the stem cells into a specific lineage. The external stimulus can be a growth factor or a second cell producing the stimulus, such as the growth factor. The effect on proliferation and differentiation can be used to elucidate the function of the proteins and determine cellular pathways.

In yet another aspect of the invention, transformed stem cells can be used to develop chimeric embryos, fetuses and animal models, such as chimeric mice, for determining the effect of various substances in vivo. Additionally, transformed stem cells can be used to produce chimeric neoplasms, such as teratomas, in mice to study the effects of various gene products on neoplasms, tumors, and the like.

In another aspect, the invention provides in vivo disease models. An embryonic stem cell transfected with a nucleic acid sequence that encodes an polypeptide can be inserted into a blastocyst to form a chimeric blastocyst. A pseudo-pregnant mouse can carry the blastocyst to term to produce a mouse that expressed the inserted gene throughout the tissues of the mouse. The source of the blastocyst can be a normal mouse, a knockout mouse, or a mouse model of human disease.

Although a number of compositions and methods similar or equivalent to those described herein can be used in the practice of the present invention, the preferred materials and methods are described herein.

Stem Cells

The stem cells for use in the invention can be any stem cell that is capable of self-replicating and can differentiate into different cell types. Thus, the stem cell can be an embryonic stem cell, a juvenile stem cell, or an adult stem cell. The stem cell can be pluripotent, multipotent, or unipotent. Moreover, the stem cells can be derived from a variety of animal species, including mammalian species such as, but not limited to, mouse, human, rat, guinea pig, sheep, goat, bovine, rabbit, canine, feline, porcine, ovine, equine and the like. A number of such stem cells are known in the art.

Adult stem cells for use in the invention can be derived from a variety of sources known in the art. For example, multipotent cells with the characteristics of stem cells have been identified in several regions of the central nervous system and at several developmental stages. These cells, often referred to as neuroepithelial stem cells (NEP cells), have the capacity to undergo self renewal and to differentiate into neurons, oligodendrocytes, and astrocytes, thus representing multipotent stem cells.

Multipotent neural stem cells can be obtained from embryonic, post-natal, juvenile, or adult neural tissue obtained from any animal that has neural tissue such as insects, fish, reptiles, birds, amphibians, mammals, and the like. Isolated liver stem cells may be obtained from fetal, juvenile, or adult liver tissue. The cells may differentiate into mature functional hepatocytes or mature bile duct cells. The stem cells that differentiate into mature functional hepatocytes are characterized by liver-specific differentiated metabolic functions, such as the expression of albumin, CCAM, glucose-6-phosphatase, and/or P450 enzyme activity.

Transfected embryonic stem cells of the invention have been demonstrated to express the secreted proteins parathyroid hormone-like protein, FrizB or sFRP3, myostatin, bone morphogenetic protein 4, insulin-like growth factor 1, neuropeptide Y, growth hormone, Wnt 2, and Wnt 11.

Transfected embryonic stem cells of the invention also stably expressed erythropoietin and IL-5. As shown in Table 1, IL-5 was detected by ELISA assay at a concentration of 461 pg/ml in an embryonic stem cell clone transfected with IL-5. Four clones transfected with erythropoietin are shown to secrete erythropoietin into the medium at concentrations ranging from 352 to 560 mU/ml.

TABLE 1
Stable Gene Expression of Erythropoietin
and IL-5 in Embryonic Stem Cells

| Erythropoietin | IL-5 |
|---|---|
| (mlU/ml | (pg/ml) |
| 352 | 461 |
| 512 | — |
| 448 | — |
| 560 | — |

The erythropoietin secreted from the stem cells maintains the physiological function of erythropoietin. It supports differentiation of erythroid precursor cells. Erythroid precursor cells isolated from human cord blood, identified as CD34+, were induced to undergo differentiation into cells of an erythrocyte phenotype after one week in coculture with erythropoietin secreted from embryonic stem cells expressing a transgene encoding erythropoietin in methylcellulose-based semi-liquid medium. The differentiating erythroid cells were identified by a red color. CD34+ cells co-cultured with the negative control, IL-5, secreted from embryonic stem cells expressing a transgene encoding IL-5 did not undergo differentiation toward an erythroid phenotype.

Proteins

In one aspect of the invention, the stem cells are transformed with a gene or part of a gene encoding a protein. The protein can be any protein, for example, a secreted protein or an extracellular domain of a transmembrane protein. The gene or part of a gene, and the proteins can be chosen from the sequence listings of PCT/US 03/27,107, PCT/US 03/27,106, PCT/US 03/26,864, and PCT/US 03/26,780, filed in the United States Receiving Office Aug. 28, 2003, and PCT/US applications entitled “Novel Human Polypeptides Encoded by Polynucleotides” attorney docket number 8940.0014.00.304, “Methods of Use for Novel Human Polypeptides Encoded by Polynucleotides” attorney docket number 8940.0016.00.304, “Human Polypeptides Encoded by Polynucleotides and Methods for their Use” attorney docket number 8940.0017.00.304, and “Novel Mouse Polypeptides Encoded by Polynucleotides” attorney docket number 8940.0018.00.304, filed in the United States Receiving Office Oct. 24, 2003, application numbers pending. These applications are incorporated herein by reference in their entirety.

Examples of proteins suitable for use in the invention include, but are not limited to, growth hormone, human growth hormone, bovine growth hormone, parathyroid hormone, parathyroid hormone-like protein, FrizB or sFRP3, myostatin, bone morphogenic protein 4, insulin-like growth factor 1, neuropeptide Y, Wnt 2, Wnt 11, thyroxin, insulin A-chain, insulin-B chain, proinsulin, relaxin A-chain, leptin receptor, fibroblast growth factor, relaxin B-chain, prorelaxin, follicle stimulating hormone, thyroid stimulating hormone, luteinizing hormone, glycoprotein hormone receptors, calcitonin, glucagon, factor VIII, an antibody, lung surfactant, urokinase, streptokinase, tissue plasminogen activator, bombesin, factor IX, thrombin, hematopoietic growth factor, tumor necrosis factor alpha, tumor necrosis factor beta, enkephalinase human serum albumin, mullerian-inhibiting substance, gonadotropin-associated peptide, β-lactamase, tissue factor protein, inhibitin, activin, vascular. endothelial growth factor, integrin receptors, thrombopoietin, protein A or D, rheumatoid factors, NGF-β, platelet growth factor, transforming growth factor, TGF-α, TGF-β, insulin-like growth factor I and II, insulin growth factor binding proteins, CD4, CD8, DNase, RNase, latency associated peptide, erythropoietin, osteoinductive factors, interferon-α, -β and -γ, colony stimulating factors, M-CSF, GM-CSF, G-CSF, stem cell factor, interleukins, IL-1, IL-2, IL-3, IL-4, IL-5, IL-6, IL-7, IL-8, IL-9, IL-10, IL-11, IL-12, superoxide dismutase, viral antigens, HIV envelope proteins, gp120, gp140, immunoglobulins, and proteins encoded by the immunoglobulin supergene family. These proteins, their ligands or receptors, and active fragments or portions of these are included as among the proteins with which the stem cells can be transformed.

Transmembrane proteins, and active fragments, analogs or variants thereof, find particular utility in generating immune responses. Thus, stem cells can be transformed with one or more nucleic acid constructs encoding the protein of interest and transformed stem cells that express the protein on their surfaces can be used to immunize a host to produce antibodies. The protein may be derived from the same species as the stem cell or from a different species. Alternatively, fragments of the stem cell, such as membrane fragments that include a transmembrane protein, can be used to immunize the subject of interest to produce antibodies.

Once produced, such antibodies can be recovered from the subject, for example from blood. Alternatively, spleen cells can be obtained from the vaccinated organism, and the recovered cells can be fused to immortalized cells to produce hybridomas, as described further below. Alternatively, mRNA can be extracted from the spleen cells and mRNA encoding an antibody of interest can be selected and used to generate cDNA molecules or a cDNA library, or to produce recombinant antibodies, also as described below.

Proteins can be produced in mini-libraries that provide sets of related proteins. For example, a mini-library of extracellular protein domains, or of secreted proteins are provided by the invention. These mini-libraries provide an efficient way of screening stem cell factors, and for producing proteins for other uses, e.g., screening.

In another aspect of the invention, the stem cells are transformed with genes encoding cytokines. Cytokines include, but are not limited to, transforming growth factor beta, epidermal growth factor family, fibroblast growth factors, hepatocyte growth factor, insulin-like growth factors, β-nerve growth factor, platelet-derived growth factor, vascular endothelial growth factor, interleukin 1 (IL-1), IL-1 receptor antagonist, IL-2, IL-3, IL-4, IL-5, IL-6, IL-6 soluble receptor, IL-7, IL-8, IL-9, IL-10, IL-11, IL-12, IL-13, IL soluble receptors, angiogenin, chemokines, colony stimulating factors, granulocyte-macrophage colony stimulating factors, erythropoietin, interferon, interferon gamma, leukemia inhibitory factor, oncostatin M, pleiotrophin, secretory leukocyte protease inhibitor, stem cell factor, tumor necrosis factors, and soluble TNF receptors. These cytokines can be obtained from human, bovine, equine, feline, canine, porcine or avian sources.

In another aspect of the invention, the stem cells are transformed with genes encoding chemokines. Chemokines are a family of relatively small proteins that have been implicated as mediators of acute and chronic inflammation, and play a role in other immunoregulatory processes. Chemokines have chemotactic properties, attracting certain cells of the immune system to sites of tissue injury and infection. Most of the α-subfamily members attract and activate neutrophils, whereas β-subfamily members attract monocytes. Certain β-subfamily members additionally have been reported to recruit basophils, eosinophils, or lymphocytes. The roles chemokines play in various disorders is discussed in Baggiolini et al. (1994) Adv. Immunol. 55:97-179.

Among the disorders believed to be mediated or exacerbated by one or more chemokines are inflammatory conditions of the lung (including inflammation associated with allergy or asthma) and skin (e.g., psoriasis). High levels of certain chemokines have been detected in the synovial fluid of inflamed joints in rheumatoid arthritis and osteoarthritis patients. The chemokine macrophage inflammatory protein-1β (MIP-1β) suppresses hematopoietic stem cell proliferation, which has been suggested to contribute to anemia in malaria patients (Burgmann et al. (1995) Clinical Immunology and Immunopathology 76:32-36. MIP-1α may play a role in the pathogenesis of experimental autoimmune encephalomyelitis (EAE), a CD4⁺ T cell-mediated inflammatory demyelinating disease of the central nervous system (Karpus et al. (1995) J. Immunol., 155:5003). The proinflammatory action of interleukin-8 (IL-8) may play a role in deleterious host responses to sepsis (Marty et al. (1994) Crit. Care Med. 22:673-679). Monocyte chemoattractant protein-1 (MCP-1), a C—C chemokine, is involved in cardiovascular disease through the recruitment of monocytes into atherosclerotic areas, and also has been implicated in fibrosis of the lung. Inhibitors of chemokines would be useful in treating the disorders discussed above. Eotaxin, another member of the C—C subfamily, is a chemo-attractant reported to be specific for eosinophils. Factors released by eosinophils contribute to hypersensitivity reactions. In the lungs of allergic asthmatic individuals, eosinophils accumulate and undergo degranulation. The resulting release of cytotoxic granule proteins aggravates lung tissue damage. The cloning of DNA encoding eotaxin, and certain functional properties of the protein, are described by Ponath et al. (1996) J. Clin. Invest., 97:604. RANTES functions as a chemo-attractant for eosinophils, monocytes, and CD45RO⁺ memory T lymphocytes (Stellato et al. (1995) J. Immunol., 155:410). By contributing to cellular recruitment in the airways during inflammation, RANTES may play a role in the pathogenesis of conditions such as asthma, rhinitis, and polyposis (Stellato et al. supra). Representative chemokines which are useful in the various embodiments of the invention include those described above, such as those that suppress myeloid cells, i.e., Macrophage Inflammatory Protein-2α (MIP-2α), Platelet Factor 4 (PF4), Interleukin-8 (IL-8), Macrophage Chemotactic and Activating Factor (MCAF), and Interferon Inducible Protein-10 (IP-10).

In another aspect of the invention, the stem cells are transformed with genes expressing the polyoma large T antigen (PyT). PyT maintains the episomal vector that carries the origin of replication of the PyT gene and an enhancer. The episomal vector carrying the heterologous nucleic acid molecule of interest can then be transfected into the ES cells. For example, plasmid pMGD20neo contains the polyoma origin of replication harboring mutated enhancer (PyF101), a modified polyoma early region that encodes the large T antigen only, and a gene that confers resistance to G418 (neo) (Gassmann et al., (1995) Proc. Natl. Acad. Sci. 92:1292-1296). After transfection the plasmid replicates in ES cells and is maintained as an extrachromosomal element. Embryonic cells that express polyoma large T antigen can be supertransfected with plasmids carrying a polyoma origin of replication, e.g., with pPyCAGIP (Chambers et al., (2003) Cell 113:645-655).

The invention encompasses randomly integrating PyT into the genome as well as integrating it into the ROSA 26 locus. The invention also encompasses the use of episomal vectors that express genes in an inducible manner. This approach can be used to circumvent potential problems in studying genes during embryogenesis or development, e.g. a position effect. The invention also encompasses a stable mouse line that has the PyT gene incorporated at the ROSA 26 locus (ROSA-PyT knock-in). The vector for targeting polyoma large T to the ROSA 26 locus has a fragment containing a truncated PyT which has been PCR amplified from plasmid pGMD20neo and cloned into a pENTR vector (Invitrogen, San Diego, Calif.) to produce plasmid pENTR-PyT. The PyT can be cloned into the targeting vector comprised of a 5′ homologous arm, SA, the PyT flanked on either side by a gateway site, bpA, PGKneobpA, a 3′homologous arm, and TK.

The ES cells with PyT can be grown in culture and used for episomal vector transfection. The episomal expression vector is constructed from a fragment of the CAG promoter (from pDRIVE-CAG, Invitrogen, San Diego, Calif.), a Gateway cassette fragment (Invitrogen, San Diego, Calif.), an IRES-hgt (hygromycin) polyA fragment (from pTITROI-MCS from Invitrogen, San Diego, Calif.). It will be cloned into a plasmid containing an ampicillin resistance gene and pUCori (derived from pcDNA3.1 from Invitrogen, San Diego, Calif.). PyF101 can be inserted into the plasmid, and the gene(s) of interest cloned between the Gateway sites. This vector can be used for in vitro screening.

Alternatively, an inducible vector can be employed in both in vivo and in vitro studies, which has an inducible promoter in the place of the CAG promoter as described above. A PyT-IRES-rtTA cassette can be constructed into the targeting vector. The source of PyT is pGMD20neo; the source of IRES is pVITRO-MCS (Invitrogen, San Diego, Calif.); the source of rtTA is BD Biosciences. Reverse tetracycline controlled transactivator (rtTA) activates transcription from the tetracycline responsive element in the presence of doxycyclin. PyT-IRES-rtTA can be targeted to the ROSA 26 locus in mouse ES cells using a targeting vector. A suitable targeting vector has a 5′ homologous arm, SA, PyT-IRES-rtTA flanked on either side by a gateway site, bpA, PGKneobpA, a 3′homologous arm, and TK. The resulting ES cells will be expanded and used for episomal vector transfection. A suitable episomal vector with a tet-responsive promoter would include the pTRE-Tight (tetracycline responsive element-Tight) (BD Sciences).

In one aspect of the present invention, stem cells are provided that have been genetically altered with DNA which encodes a protein or polypeptide which is believed to promote differentiation of the cell into a specific cell line. The DNA which encodes a protein or polypeptide which promotes differentiation of the embryonic stem cell into a specific cell line is DNA encoding a protein or polypeptide normally found in the specific differentiated cell line, and is preferably generally not present in other types of cells. In one aspect, the DNA which encodes a protein or polypeptide which promotes differentiation of the embryonic stem cell into a specific differentiated cell line is DNA encoding a growth factor or a transcription factor present in the specific cell line to promote differentiation of the cell into the specific cell line. In another aspect, the DNA encoding a transcription factor is DNA encoding a transcription factor present in neuronal cells, and the specific cell line is a neural stem cell line. In another aspect, the DNA encoding a growth hormone or a transcription factor is DNA encoding a transcription factor, such as the MyoD gene, present in muscle cells, and the specific cell line is a muscle cell line. In yet another aspect, the DNA encoding a growth hormone or a transcription factor is DNA encoding a transcription factor present in hematopoietic cells, and the specific cell line is a hematopoietic stem cell line.

In another aspect, the stem cells are genetically modified with genes that are preferentially or exclusively expressed in particular tissue types. For example, genes that are preferentially or exclusively expressed within the nervous system include the following: Nova-1, Nova-2, N-type calcium channels, GABA(A) receptor, dopamine receptors, agrin, neurexins, synapsins, PPT, CaM, vacuolar H⁺-ATPase subunit B (isoform H057), renin, nestin, GFAP, and neurofilament H. Genes that are preferentially or exclusively expressed within epithelia include E-cadherin and estrogen receptor (ER)3. The gene flk1 is preferentially expressed in the vascular endothelium. Genes that are preferentially or exclusively expressed within the endoderm include TTF1/Nkx2.1, Nkx2.6, Pax8, Pax9, Hex1, Hoxb1, Pdx1, Pax4, Pax6, Nkx2.2, Is1-1, NeuroD, cdx2, Hoxd genes, pancreas amylase 2, pancreas PDX-1, and pancreatic insulin. Genes that are preferentially or exclusively expressed within cardiac, skeletal, and muscle tissue include cartilage matrix protein, collagen II adult type, myotonin protein kinase gene, TEF-1, cardiac alpha actin, cardiac myosin heavy chain-alpha (MHC alpha), cardiac myosin heavy chain-beta (MHC beta), myosin light chain-1A (MLC1A), myosin light chain-1V (MLC1V), α-tropomyosin (α-TM), cardiac troponin-T (Ctnt), atrial natriuretic factor (ANF), cytochrome C oxidase (COX) tissue-specific isoforms (VIa, VIIa, VIII), Hand1, FHL2, hCsx, calcitonin receptor-like protein, and aldosterone-synthase. Genes that are preferentially or exclusively expressed within the pancreas, liver, or prostate include the following: albumin, alpha-fetoprotein, α1-antitrypsin, pancreas amylase 2, pancreas PDX-1, pancreatic insulin, hB1f (human B1-binding factor), kallikrein (KLK) gene clusters, apolipoprotein (a), plasminogen, insulin-like growth factor binding protein 1 (IGFBP-1), phenylalanine hydroxylase (PAH), S-adenosylmethionine synthetase (SAMS), transthyretin, tyrosine aminotransferase, glucose-6-phosphatase, dipeptidylpeptidase IV, cytokeratin 19, biliary glycoprotein, γ-glutamyltranspeptidase, vinculin, cytokeratin 18, cytokeratin 8, c-met, Gata-6, Gata-4, variant hepatocyte nuclear factor 1, hepatocyte nuclear factor I-α, hepatocyte nuclear factor 4-α1, hepatocyte nuclear factor 4-α7, hepatocyte nuclear factor 3-α, hepatocyte nuclear factor 3-β, hepatocyte nuclear factor 3-γ, apolipoprotein B, Smad-4, evx-1, contrapsin, major urinary proteins, α-1-microglobulin/bikunin precursor gene, phosphoenolpyruvate carboxykinase, carbamoylphosphate synthetase I, inter-α1-trypsin inhibitor, α1 acid glycoprotein, haptoglobin, vitamin D-binding protein, ceruloplasmin, fibrinogen, α2-macroglobulin, thiostatin, transferrin, and retinol-binding protein.

In yet another aspect of the invention, the stem cells are genetically modified by incorporation of nucleic acids that encode for more than one protein. Thus, DNA encoding for 2, 3, 4, 5, 10, 15, or 20 different proteins can be used to transfect the stem cells. The plurality of proteins can be related, for example, they may be within the same selected family of proteins such as growth factors, transmembrane proteins, kinases, proteases, or phosphatases, or may be involved with a common differentiation pathway or genes expressed within a particular cycle of stem cell differentiation. Alternatively, the plurality of proteins may not be related.

In yet another aspect of the invention, the stem cells are genetically modified by incorporation of a library of nucleic acids. The library of nucleic acids can be prepared by methods known in the art.

Vectors

The DNA sequences encoding the proteins can be obtained from natural sources, such as an organism or tissue sample for example, or can be synthetically produced using sequences obtained from the literature or from publicly accessible databases. For example, the DNA sequence for erythropoietin is disclosed by Jacobs et al. (1985) Nature 313:806; Lin et al. (1985) Proc. Natl. Acad. Sci. 82:7580 (1985); Krantz (1991) Blood 77:419; and Dube et al. (1988) J. Biol. Chem. 263, 17516. The DNA sequence for G-CSF is disclosed by Nagata et al. (1986) EMBO J. 5:575 and Nagata et al. (1986) Nature 319:415. The DNA sequence for GM-CSF is disclosed by Gough et al. (1984) Nature 309:763 and Nicola et al. (1979) J. Biol. Chem. 254:5290. The DNA for IL-3 is disclosed by Yang et al. (1986) Cell 47:3. The DNA for leukemia inhibitory factor (LIF) is disclosed by Metcalf (1991) Int. J. Cell Clon. 9:85 and Sutherland et al. (1989) Leuk. 3:9. DNA sequence for IL-11 is disclosed by Kawashima et al. (1994) FEBS Lett. 283:199 and Paul et al. (1990) Proc. Natl. Acad. Sci. 87:7512. The DNA for thrombopoietin is disclosed by de Sauvage et al. (1994) Nature 369:533 and Kaushansky et al. (1994) Nature 369:568.

Alternatively, the gene which promotes differentiation and/or proliferation of the stem cell into a specific cell line may be isolated using standard genetic engineering techniques (such as, for example, by isolating such DNA from a cDNA library of the specific cell line) and placed into an appropriate expression vector, which then is transformed into the stem cells. Thus, the stem cells may be genetically-altered by the introduction of heterologous DNA. A genetically-altered stem cell is one into which has been introduced, by means of recombinant DNA techniques, such as homologous recombination, a gene, as described above. The stem cell can be altered with full-length gene sequences, for example sequences encoding the proteins described above, or with cDNA or fragments thereof, where cDNA is DNA separated from the 5′ and 3′ coding sequences with which it is immediately contiguous in the naturally occurring genome of an organism. A genetically-altered stem cell may contain DNA encoding a protein under the control of a promoter that directs strong expression of the protein.

The genetic modification of the stem cells can be performed by transfection using methods known in the art including CaPO₄ transfection, DEAE-dextran transfection, by protoplast fusion, electroporation, lipofection, and the like. With direct DNA transfection, cells can be modified by particle bombardment, receptor mediated delivery, and cationic liposomes.

In another aspect of the invention, the stem cells can be altered by the introduction of the full-length gene sequences of the proteins. The full-length gene sequences can be isolated from vectors or synthesized completely or in part using various oligonucleotide synthesis techniques known in the art, such as site-directed mutagenesis and polymerase chain reaction (PCR) techniques where appropriate. In particular, one method of obtaining nucleotide sequences encoding the desired sequences is by annealing complementary sets of overlapping synthetic oligonucleotides produced in a conventional, automated polynucleotide synthesizer, followed by ligation with an appropriate DNA ligase and amplification of the ligated nucleotide sequence via PCR. See, e.g., Jayaraman et al. (1991) Proc. Natl. Acad. Sci. USA 88:4084-4088. Additionally, oligonucleotide directed synthesis (Jones et al. (1986) Nature 54:75-82), oligonucleotide directed mutagenesis of pre-existing nucleotide regions (Riechmann et al. (1988) Nature 332:323-327 and Verhoeyen et al. (1988) Science 239:1534-1536), and enzymatic filling-in of gapped oligonucleotides using T₄ DNA polymerase (Queen et al. (1989) Proc. Natl. Acad. Sci. USA 86:10029-10033) can be used to provide the sequences.

Once coding sequences have been prepared or isolated, such sequences can be cloned into any suitable vector or replicon. Numerous cloning vectors are known to those of skill in the art, and the selection of an appropriate cloning vector is a matter of choice. Suitable vectors include, but are not limited to, plasmids, phages, transposons, cosmids, chromosomes or viruses which are capable of replication when associated with the proper control elements.

The coding sequence is then placed under the control of suitable control elements, depending on the system to be used for expression. Thus, the coding sequence can be placed under the control of a promoter, ribosome binding site, and, optionally, an operator, so that the DNA sequence of interest is transcribed into RNA by a suitable transformant. The coding sequence may or may not contain a signal peptide or leader sequence which can later be removed by the host in post-translational processing. See, e.g., U.S. Pat. Nos. 4,431,739; 4,425,437; 4,338,397.

An expression vector for the present invention can be constructed by any conventional methods. For example, the expression vector can be constructed such that the gene of interest is located in the vector under the control of the appropriate regulatory sequences. Modification of the sequences encoding the gene of interest may be desirable to achieve this end. For example, in some cases it may be necessary to add to the coding sequence of the gene of interest so that it can be attached to the control sequences in the correct reading frame. The control sequences and other regulatory sequences may be ligated to the coding sequence prior to insertion into a vector. Alternatively, the coding sequence can be cloned directly into an expression vector which already contains the control sequences and an appropriate restriction site. Several possible vector systems are available and known in the art. Some vectors use DNA elements which provide autonomously replicating extra-chromosomal plasmids, generally derived from animal viruses. Other vectors include Vaccinia virus expression vectors. Still other vectors integrate the desired polynucleotide into the host chromosome.

The genetically modified stem cells can be selected by introducing one or more markers (e.g., an exogenous gene) which allows for the selection of cells which contain the expression vector. The selectable marker gene can either be directly linked to the DNA sequences to be expressed, or introduced into the same cell by co-transformation. Additional elements may also be needed for optimal synthesis of mRNA. These elements may include splice signals, as well as transcription termination signals.

A number of selection systems may be used, including, but not limited to, the herpes simplex virus thymidine kinase, hypoxanthine-guanine phosphoribosyltransferase, and adenine phosphoribosyltransferase genes can be employed in tk⁻, hgprt⁻ or aprt⁻ cells respectively. Also, antimetabolite resistance can be used as the basis of selection for dhfr, which confers resistance to methotrexate; gpt, which confers resistance to mycophenolic acid; neo, which confers resistance to the aminoglycoside G-418; and hygro, which confers resistance to hygromycin genes. Additional selectable genes have been described, such as trpB, which allows cells to utilize indole in place of tryptophan; hisD, which allows cells to utilize histinol in place of histidine; and ODC (ornithine decarboxylase) which confers resistance to the ornithine decarboxylase inhibitor, 2-(difluoromethyl)-DL-ornithine, DFMO.

Other markers useful herein include cell surface markers such as alkaline phosphatase, nerve growth factor receptor, or any other suitable membrane-associated moiety. Representative examples of such markers and associated prodrug molecules include alkaline phosphatase and various toxic phosphorylated compounds such as phenolmustard phosphate, doxorubicin phosphate, mitomycin phosphate and etoposide phosphate; β-galactosidase and N-[4-(β-D-galactopyranosyl) benyloxycarbonyl]-daunorubicin; azoreductase and azobenzene mustards; β-glucosidase and amygdalin; β-glucuronidase and phenolmustard-glucuronide and epirubicin-glucuronide; carboxypeptidase A and methotrexate-alanine; cytochrome P450 and cyclophosphamide or ifosfamide; DT diaphorase and 5-(aziridine-1-yl)-2,4,dinitrobenzamide (CB1954) (Cobb et al. (1969) Biochem. Pharmacol 18:1519, Knox et al. (1993) Cancer Metastasis Rev. 12:195); β-glutamyl transferase and β-glutamyl p-phenylenediamine mustard; nitroreductase and CB1954 or derivatives of 4-nitrobenzyloxycarbonyl; glucose oxidase and glucose; xanthine oxidase and hypoxanthine; and plasmin and peptidyl-p-phenylenediamine-mustard. Nonimmunogenic markers may also be made by expressing an enzyme in a compartment of the cell where it is not normally expressed.

Still other suitable markers are genes which impart color to those cells transfected with a nucleic acid element containing the selectable marker such that detection can be achieved by virtue of a color change (either visible or fluorescent). For example, the gene encoding luciferase can be used as the selectable marker. Similarly, the gene encoding Green Fluorescent Protein (GFP) or derivatives thereof such as Enhanced Green Fluorescent Protein (EGFP), and like molecules, can be used. These and other selectable markers can be obtained from commercially available plasmids, using techniques well known in the art. See, e.g., Sambrook et al., supra.

In one aspect, DNA encoding the protein of interest can be introduced into the stem cells by the method of Remy et al. (1995) Proc. Natl. Acad. Sci. USA 92(5): 1744-8, which is a modular transfection system based on lipid-coating the polynucleotides. The particle core is composed of the lipopolyaminc-condensed polynucleotide in an electrically neutral ratio to which other synthetic lipids with viral properties are hydrophobically adsorbed. Usually a zwitterionic lipid, such as dioleoyl phosphatidylethanolamine, can be used to coat the nucleotides.

Another targeted delivery system for the polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macromolecules. RNA, DNA and intact virions can be encapsulated within the aqueous interior and be delivered to mammalian cells, plant, yeast and bacterial cells (Fraley et al. (1981) Trends Biochem. Sci., 6:77). The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine, and distearoylphosphatidylcholine.

In another aspect of the invention, viral vectors are used to transfect the stem cells with the genes encoding the proteins. Viral vectors include retroviruses (including lentiviruses), adenoviruses, adeno-associated viruses and herpes simplex virus type I. Such vectors may additionally require helper cell lines for replication and stem or differentiated cell specific regulatory sequences. Virus vectors that carry a heterologous gene (transgene) generally will contain viral, for example retroviral long terminal repeat (LTR), simian virus 40 (SV40), cytomegalovirus (CMV); or liver (such as the albumin promoter; see, Connelly et al., Hum. Gene Ther. 6(2): 185-93 (1995) and Milos & Zaret, Genes Dev. 6(6):991-1004 (1992)) or pancreatic cell-specific promoters (such as insulin promoters).

As will be evident to one of skill in the art, the DNA sequence encoding a protein or a fragment of a protein can be targeted to any locus within the genome of the stem cell. In one aspect of the invention, the locus can be selected such that it has a higher targeting frequency, is not hypo-insufficient, and is capable of ubiquitously expressing the inserted DNA at high frequency. Thus, the choice of the locus will depend on the source of the stem cell and the method of transfection. For example, if mouse ES cells are selected for use in the invention and mouse ES cells are selected to be genetically modified using homologous recombination, then the ROSA 26 locus can be targeted for the incorporation of the DNA sequences. Any gene loci can be used in the practice of the invention provided targeting one copy of the gene will not result in a haploinsufficient phenotype. Thus, the locus can be ROSA 26, ROSA 5, ROSA 11, G3BBP(BT5), phosphoglycerate kinase, actin loci, and the like.

Proliferation and Differentiation

Methods for the proliferation and differentiation of various types of stem cells are known in the art. Typically, the embryonic stem cells are cultured in a standard culture medium (such as, for example, Minimal Essential Medium), which may include supplements e.g., glutamine, and β-mercaptoethanol. The medium may also include leukemia inhibitory factor (LIF), or factors with LIF activity, e.g., CNTF or IL-6, and horse serum. LIF and factors with LIF activity prevent spontaneous differentiation of the embryonic stem cells, and are removed prior to the addition of an agent which promotes or stimulates differentiation. Horse serum promotes differentiation of the embryonic stem cells into the specific cell type after the addition of the agent to the medium. After the cells have been cultured for a period of time sufficient to permit the cells to proliferate to a desired number, the cells are washed free of LIF, and then cultured under conditions which provide for the growth of the cells at a decreased growth rate but which also promote differentiation of the cells. The cells are cultured in the presence of an agent which promotes or stimulates differentiation of the embryonic stem cells into a desired cell line, and in the presence of fetal bovine serum at a concentration of from about 5% by volume to about 10% by volume, preferably at about 10% by volume. The presence of the fetal bovine serum at a concentration of from about 5% by volume to about 10% by volume, and of the agent, provides for growth or proliferation of the cells at a rate which is less than the optimal rate, while favoring the differentiation of the cells into a homogeneous desired cell type. The desired cell type is dependent upon the agent which promotes or stimulates the differentiation of the embryonic stem cells.

The embryonic stem cells can also be cultured in a three-dimensional format. For example, they may be placed in a culture vessel to which the cells do not adhere, e.g., polystyrene or glass. The substrate may be untreated, or may be treated such that a negative charge is imparted to the cell culture surface. In addition, the cells may be plated in methylcellulose in culture media, or in normal culture media in hanging drops. Media which contains methylcellulose is viscous, and the embryonic stem cells cannot adhere to the dish. Instead, the cells remain isolated, and proliferate, and form aggregates. In order to form aggregates in hanging drops of media, cells suspended in media are spotted onto the underside of a lid of a culture dish, and the lid then is placed on the culture vessel. The cells, due to gravity, collect on the undersurface of the drop and form aggregates.

The stem cells can then be differentiated into a specific cell line. In one aspect, the differentiation is initiated by the expression of the polynucleotide sequences used to transfect the stem cells. In another aspect, the differentiation can be initiated by the addition of factors that are known to cause differentiation. For example, SCL controls hematopoietic stem cell differentiation (Porcher et al. (1996) Cell 86:47-57) and neurogenic stem cell differentiation can be controlled by the BHLH proteins MASH, neurogenin, and neuro D (reviewed in Morrison et al. (1997) Cell 88:287-298 and Andersen (1994) FASEB J., 8:707-713). Liver stem cell differentiation can be regulated by a combination of transcription factors including NF-κB, Stat3, and C/EBP (Taub (1996) FASEB J. 10:413-427).

Other factors are known to those of skill in the art. For example, differentiation into the hematopoietic lineage is discussed in Wiles, M. Embryonic Stem Cell Differentiation in vitro (1993) Meth. Enzymol. 225:900-918; myogenic differentiation is discussed in Prelle et al. (2000) Biochem. Biophys. Res. Commun. 227(3):631-638; differentiation into neuronal lineage is discussed in Farinas et al. (2000) Brain Res. Bull. 57(6):809-16, and Strubing et al. (1995) ALTEX. 12(3):129-137.

Antibodies

As explained above, the transformed stem cells of the present invention can be used to produce antibodies in vivo which in-turn can be used for screening, diagnostic, and/or therapeutic purposes. Alternatively, fragments of the stem cell, such as membrane fragments that include a transmembrane protein, can be used to immunize the subject of interest to produce antibodies. Additionally, nucleic acid can be extracted from cells or tissues of the immunized host organism, such as from blood and spleen, and molecules coding for antibodies specific for the protein molecule of interest, can be used to generate antibodies in vitro to produce the desired antibody or fragments thereof. The extracted nucleic acid can also be used to create a cDNA library for, e.g., screening purposes. Antibodies isolated from spleen can also be used to generate monoclonal antibodies as described below.

Antibodies are produced by administering stem cells that have been transformed with a gene of interest, such as a gene encoding a transmembrane protein, to a host animal and then isolating the antibodies, or isolating RNA encoding the antibodies, from the host organism. One convenient way of obtaining antibodies or RNA encoding antibodies so produced is by isolating the same from host cells such as spleen cells.

Particularly, the antibodies may be polyclonal or monoclonal, may be a human antibody, or may be a hybrid or chimeric antibody, such as a humanized antibody, an altered antibody, F(ab′)2 fragments, F(ab) fragments, Fv fragments, a single-domain antibody, a dimeric or trimeric antibody fragment construct, a minibody, or functional fragments thereof with the desired specificity. Antibodies are produced using techniques well known to those of skill in the art and disclosed in, for example, U.S. Pat. Nos. 4,011,308; 4,722,890; 4,016,043; 3,876,504; 3,770,380; and 4,372,745.

Antibody fragments which retain the ability to recognize the molecule of interest, will also find use in the subject invention. A number of antibody fragments are known in the art which comprise antigen-binding sites capable of exhibiting immunological binding properties of an intact antibody molecule. For example, functional antibody fragments can be produced by cleaving a constant region, not responsible for antigen binding, from the antibody molecule, using e.g., pepsin, to produce F(ab′)2 fragments. These fragments will contain two antigen binding sites, but lack a portion of the constant region from each of the heavy chains. Similarly, if desired, Fab fragments, comprising a single antigen binding site, can be produced, e.g., by digestion of polyclonal or monoclonal antibodies with papain. Functional fragments, including only the variable regions of the heavy and light chains, can also be produced, using standard techniques such as recombinant production or preferential proteolytic cleavage of immunoglobulin molecules. These fragments are known as FV. See, e.g., Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

A single-chain Fv (“sFv” or “scFv”) polypeptide is a covalently linked VH-VL heterodimer which is expressed from a gene fusion including VH- and V L-encoding genes linked by a peptide-encoding linker. Huston et al. (1988) Proc. Nat. Acad. Sci. USA 85:5879-5883. A number of methods have been described to discern and develop chemical structures (linkers) for converting the naturally aggregated, but chemically separated, light and heavy polypeptide chains from an antibody V region into an sFv molecule which will fold into a three dimensional structure substantially similar to the structure of an antigen-binding site. See, e.g., U.S. Pat. Nos. 5,091,513, 5,132,405 and 4,946,778. The sFv molecules may be produced using methods described in the art. See, e.g., Huston et al. (1988) Proc. Nat. Acad. Sci. USA 85:5879-5883; U.S. Pat. Nos. 5,091,513, 5,132,405 and 4,946,778. Design criteria include determining the appropriate length to span the distance between the C-terminus of one chain and the N-terminus of the other, wherein the linker is generally formed from small hydrophilic amino acid residues that do not tend to coil or form secondary structures. Such methods have been described in the art. See, e.g., U.S. Pat. Nos. 5,091,513, 5,132,405 and 4,946,778. Suitable linkers generally comprise polypeptide chains of alternating sets of glycine and serine residues, and may include glutamic acid and lysine residues inserted to enhance solubility.

“Mini-antibodies” or “minibodies” will also find use with the present invention. Minibodies are sFv polypeptide chains which include oligomerization domains at their C-termini, separated from the sFv by a hinge region. Pack et al. (1992) Biochem 31:1579-1584. The oligomerization domain comprises self-associating α-helices, e.g., leucine zippers, that can be further stabilized by additional disulfide bonds. The oligomerization domain is designed to be compatible with vectorial folding across a membrane, a process thought to facilitate in vivo folding of the polypeptide into a functional binding protein. Generally, minibodies are produced using recombinant methods well known in the art. See, e.g., Pack et al. (1992) Biochem 31:1579-1584; Cumber et al. (1992) J Immunology 149B: 120-126.

Screening

The stem cell libraries of the invention can be used in assays for screening, testing and comparing agents or libraries of agents. The agents can be genes, proteins, peptides, small molecules, and the like, and any convenient multiplex testing configuration can be used. One convenient configuration is a 96-well microtiter dish, for example, although dishes with a greater or lesser number of wells can also be used in the practice of the invention. In one configuration, a first population of stem cells is placed in one well, a second population of stem cells is placed in a second well, a third population of stem cells is placed in a third well, and so on, for up to any desired number of stem cell populations, such as 5, 10, 25, 50, 100, 200, 300, 1000, 2000, or more. In experiments where greater than 96 stem cell populations are used, other microtiter dishes that have more wells, such as 192, 288, 386, and the like can be used, or multiple plates can also be used. Each stem cell population contains stem cells transformed with a vector containing a gene of interest. In one aspect, the gene of interest is different for the different stem cell populations in the library. Thus, for example, a configuration of n stem cell populations can be transformed with up to n different genes of interest. In another aspect, each well can contain a plurality of stem cell populations. Thus, each well can have, e.g., about 1, 2, 3, 4, or 5 different stem cell populations, with each population containing stem cells transformed with a vector containing a different gene of interest. In yet another aspect, each stem cell population contains stem cells transformed with a plurality of genes of interest, and each well can contain a population of such cells.

The library can then be used to study the effect of an agent on the stem cell's growth or differentiation. This effect can be modulation in the growth or differentiation of the stem cells in the library, such as, for example, enhanced growth or differentiation or inhibition of growth or differentiation. In another aspect, the gene of interest is the same for the different stem cell populations. In the latter case, the library can be used to detect combination effects that is the effect of the gene of interest as well as any additional factors or cells, on the growth or differentiation of the stem cells in the library. The additional factors include, for example, factors that are in solution or factors that are secreted by cells or that are present as extracellular portions of transmembrane proteins on the surface of other cells added to the library of stem cells. In another aspect, the stem cell libraries of the invention can be used to study the effect on another cell type. For example, some novel secreted molecules are implicated in axon guidance during development of the nervous system. ES cells expressing these secreted molecules can be used as the factor source to see if these molecules have repellent or/and other activities in root dorsal ganglia explant system.

In one aspect of the invention, the activity of the proteins or fragments thereof encoded by the nucleic acids used to transfect the stem cells can be assayed. In this aspect, the gene encoding the protein is expressed, and the modulation of the proliferation and/or differentiation of the stem cell library transformed with the gene are observed. Thus, changes in the rate of proliferation, the lack of proliferation, and/or differentiation of the genetically-altered stem cells can be compared with the wild or non-genetically-altered stem cells. For example, mouse ES cells, such as the mouse 129/SvJ cell line, are derived from the early mouse embryo and are grown under culture conditions well known in the art. The vector is introduced into ES cells by transformation methods such as electroporation, liposome delivery, microinjection, and the like, which are well known in the art. The vector can contain genes for a secreted protein, for example, such as growth factors or cytokines. The endogenous rodent gene is replaced by the disrupted disease gene through homologous recombination and integration during cell division. Then transformed ES cells are selected and used to study the proliferation and differentiation into various cell types. of various cell types and tissues in vitro, such as neural cells, hematopoietic lineages, and cardiomyocytes (Bain et al. (1995) Dev. Biol. 168:342-357; Wiles and Keller (1991) Development 111:259-267; and Klug et al. (1996) J. Clin. Invest. 98:216-224).

In another aspect, the transformed stem cells or a library of the transformed stem cells can be differentiated into a different cell type by the addition of factors that promote differentiation. The additional differentiation promoters can be lineage specific or non-lineage specific, and can be supplied individually, in a formulation containing a combination of factors, or by the addition of a cell or cells that are capable of providing the differentiation factors to the genetically-modified stem cell. The differentiated cell can be identified by the marker on the surface of the cell or by its phenotype. For example, the transformed ES cells described above are selected, exposed to an exogenously added factor, and the proliferation and differentiation of the stem cell populations into various cell types and tissues in vitro, such as neural cells, hematopoietic lineages, and cardiomyocytes (Bain et al. (1995) Dev. Biol. 168:342-357; Wiles and Keller (1991 ) Development 111:259-267; and Klug et al. (1996) J. Clin. Invest. 98:216-224) is studied.

In another aspect of the invention, a combination of genetically modified stem cells, such as a library of ES cells expressing different growth factors, can be co-cultured. The synergistic effects of the different growth factors on the proliferation and differentiation can be assayed as described in the Examples. In one aspect of the invention, the ES cells can be directly in contact with each other. In this aspect, about 2, 3, 4, 5, 10, 15, 20, 25, 50, 100, 200, 500, 1000, or more stem cells, with each stem cell expressing a different protein, such as a growth factor, can be placed in the same well and allowed to proliferate and differentiate. In another aspect, the stem cells comprising the library can be co-cultured but be spatially separated. In this aspect, the stem cells are preferably in communicative contact with each other. For example, the stem cells can be separated by a membrane where the membrane permits the diffusion of small molecules and proteins but not cells. In yet another aspect, the library of stem cells can be allowed to proliferate and differentiate, either in direct contact or in indirect contact, the supernatants from the library of stem cells expressing various excreted molecules collected, and the collected molecules used to characterize their effect on other cells, such as T cell or B cell growth or inhibition. In an aspect, the stem cell library can be used to determine a pharmaceutical property of an agent, or to rank-order the pharmacological properties of a library of agents. The pharmacological property can be, for example, potency, efficacy, and the like. A library of agents can be screened to determine the ability of the library or each agent in the library to elicit a dose-dependent and/or time-dependent effect on the stem cell library. The method typically employs a library of stem cells that are cultured under substantially identical conditions, such as, for example, in the wells of a multiwell culture vessel (e.g., 96 well microtitre dish). To each well is added a predetermined concentration of an agent or a library of agents such that a plurality of concentrations are represented in each well.

For example, a 96-well plate may have each row representing a different agent and each column representing a series of predetermined different concentrations for each agent. The stem cell library can be allowed to proliferate and/or differentiate in the presence of the agent(s) for a period of time. Following or during the incubation time period, the extent of proliferation, lack of proliferation, degree of differentiation, and/or expression of cell surface reporter proteins can be detected for each well and compared to a control well containing stem cells libraries cultured under substantially identical conditions without the agent. Optionally, the extent of proliferation, lack of proliferation, degree of differentiation, and/or expression of cell surface reporter proteins in each well can be detected and quantified at a plurality of time points to create a dose-response curve or follow time dependent response of any other pharmacological parameter. Thus, each agent or library of agents can be rank-ordered relative to each other, and agent having the desired pharmacological profile can be identified.

Transformed stem cells can also be used in methods of determining gene function in vivo. For example, a gene of interest can be used to target a specific locus in an ES cell, e.g., a locus described above, such as the ROSA 26 locus. The transformed stem cell can be injected into an embryonic precursor, such as a blastocyst, using standard techniques, and the blastocyst can be implanted into the uterus of an animal, e.g., a non-human animal, such as a mouse, by methods well known in the art. The blastocyst can then be allowed to develop into a chimeric embryo and chimeric fetus in vivo, and ultimately, a chimeric animal can be produced, such as a chimeric mouse. Preferably, the chimeric embryo, fetus, or animal produces the product encoded by the gene of interest in multiple tissues, such that the effect of the gene product on the embryo, fetus, or animal, can be determined. Cell lines can be produced from cells or tissues obtained from the chimeric embryo, fetus, or animal above.

Alternatively, a gene of interest can be used to target a specific locus in an ES cell, e.g., a locus described above, such as the ROSA 26 locus, and the transformed embryonic stem cell can be provided to a tissue of an animal, for example, delivered to an immunocompromised animal such as a nude mouse, and the embryonic stem cell can then develop into a chimeric neoplasm, such as a teratoma. The effect of the gene product on the neoplasm can be determined, thus providing information on the action of particular agents on cancerous cells, precancerous cells, and the like. Cell lines can also be developed from the chimeric neoplasm above.

In Vivo Disease Models

The transformed stem cells of the invention can be used to develop in vivo mouse models of human disease. In general, a gene construct encoding a polypeptide is inserted into the mouse embryonic stem cells to produce transfected stem cells. One or more than one polypeptide can be encoded by the construct. The polypeptide can be, e.g., a secreted protein, a fragment of a secreted protein, a transmembrane protein, an extracellular domain of a transmembrane protein, or a combination of these. The gene construct is inserted in a locus that allows the gene to be expressed in all tissues of the mouse.

The resulting transfected stem cells are inserted into a blastocyst, e.g., at the 64 cell stage to form a chimeric blastocyst. Normal mice, knockout mice, or mouse models of human disease can provide a source for these blastocysts. When implanted into a pseudo-pregnant mouse, the blastocysts can develop into chimeric embryos, fetuses, and mice.

Mouse models that are useful for practicing the invention include, but are not limited to, mice that overexpress Aβ peptide, overexpress TGFβpeptide, or carry a mutation that cause Parkinson's disease. Other useful mouse models include the SCID mouse, non-obese diabetic mouse, Rb−/− mouse, and p53 −/− mouse. These models provide an opportunity to observe whether an inserted gene corrects the deficiency. The chimeric mice can also be produced by breeding, e.g., by crossing a mouse carrying a gene of interest from the library with a mouse model of human disease.

Specifically, the invention provides a system for conducting in vivo and in vitro testing of secreted protein function, for expression or manufacture of proteins. The system provides targeting a gene to a locus, e.g., the ROSA 26 locus in mouse ES cells and allowing the transfected DNA to proliferate and differentiate in vitro. The ROSA 26 locus directs the ubiquitous expression of the heterologous gene (Soriano et al., U.S. Pat. No. 6,461,864). For example, the effect of the transfected DNA on differentiated or undifferentiated cells can be monitored in vitro. Differentiation of cells, e.g., cardiomyocytes, hepatocytes, skeletal myocytes, etc. can be monitored by morphologic, histologic, and/or physiologic criteria.

The transfected ES cells can be added to a blastocyst, which can then be implanted into a pseudopregnant mouse to produce a mouse useful for the study of the effect of the transfected gene on the individual tissues of the mouse. The tissues can be isolated and studied, or cells and/or cell lines can be isolated from the tissues and studied. For example, ES cells transfected with IL-5 and incorporated into a blastocyst produced a chimeric mouse that expressed a greater than normal number of eosinophils in the liver. This is a previously observed effect of IL-5, and demonstrates that the ES cell mouse expression system (ESpresso mouse) can be used to determine the function of unknown and novel secreted polypeptides. Mice possessing phenotypic changes as a result of transgene expression may physically appear only slightly chimeric.

While the present invention has been described with reference to the specific embodiments thereof, it should be understood by those skilled in the art that various changes may be made and equivalents may be substituted without departing from the true spirit and scope of the invention. In addition, many modifications can be made to adapt a particular situation, material, composition of matter, process, process step or steps, to the objective, spirit and scope of the present invention. All such modifications are intended to be within the scope of the claims appended hereto.

It is to be understood that both the foregoing general description and the following detailed description are exemplary and explanatory only and are not restrictive of the invention, as claimed. Moreover, it must be understood that the invention is not limited to the particular embodiments described, as such may, of course, vary. Further, the terminology used to describe particular embodiments is not intended to be limiting, since the scope of the present invention will be limited only by its claims.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test the invention.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

It must be noted that, as used herein and in the appended claims, the singular forms “a,” “or,” and “the” include plural referents unless the context clearly dictates otherwise. Thus, for example, reference to “a subject polypeptide” includes a plurality of such polypeptides and reference to “the agent” includes reference to one or more agents and equivalents thereof known to those skilled in the art, and so forth.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

The specification is most thoroughly understood in light of the cited references, all of which are hereby incorporated by reference in their entireties. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### EXAMPLES

The examples, which are intended to be purely exemplary of the invention and should therefore not be considered to limit the invention in any way, also describe and detail aspects and embodiments of the invention discussed above. The examples are not intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Designing Targeting Vectors for Insertion into Cells of the Library

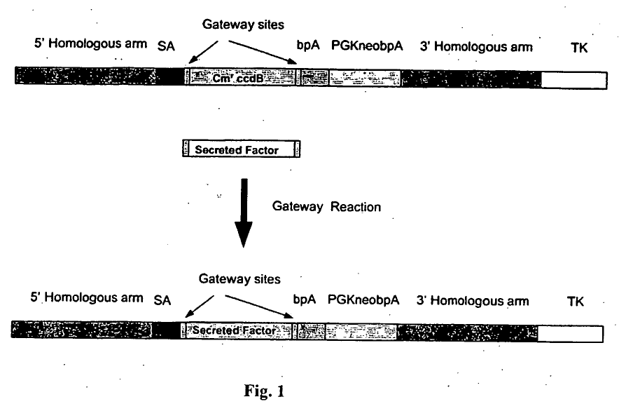

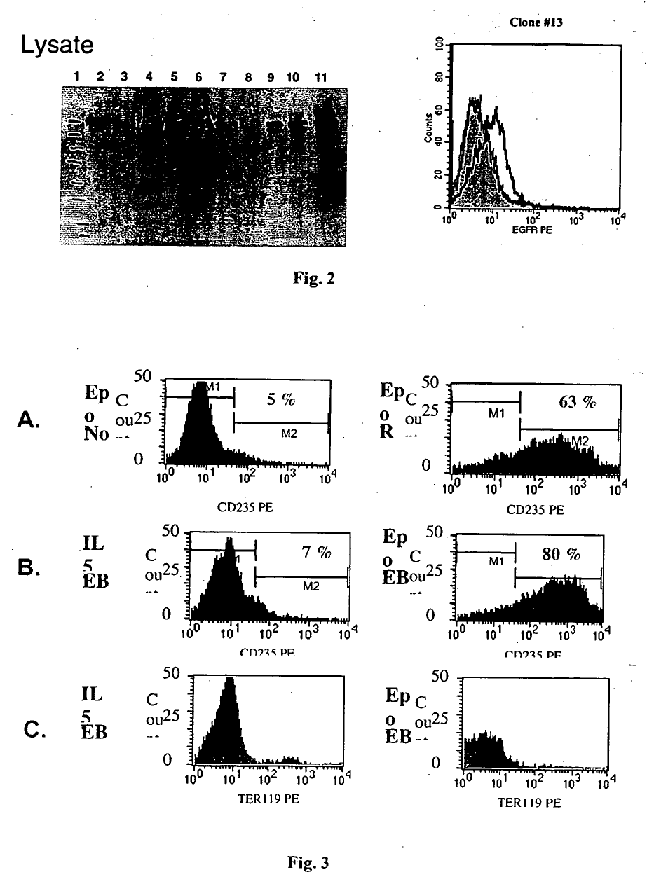

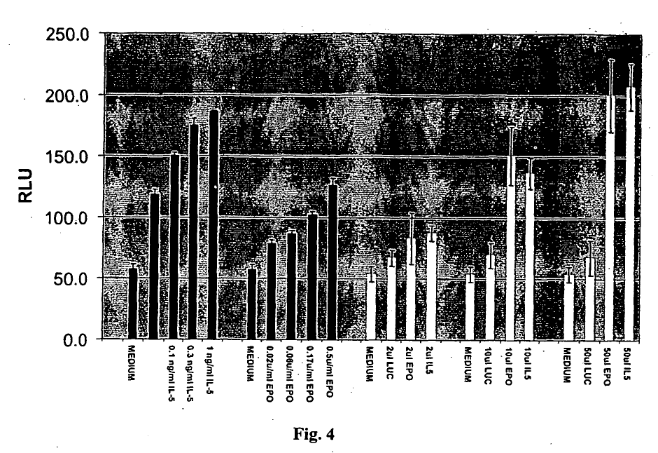

A targeting vector for secreted or other molecules targeting to the ROSA26 locus was constructed as shown in FIG. 1. The PGKneobpA fragment was made by combining PGKneo from New England Biolabs (Beverly, Mass.) and bovine growth hormone poly A (bpA) from BD Biosciences Clontech (Palo Alto, Calif.). The adenovirus major late transcript splicing acceptor (SA) was PCR amplified from adenovirus genomic DNA. TK gene was PCR amplified from a cosmid vector svPHEP from ATCC (Manassas, Va.). The 5′ and 3′ homologous arms were PCR amplified and cloned from genomic DNA according to a public genomic database, e.g., NCBI. As read from the 5′ to 3′ direction, the basic targeting vector without the gene of interest was made by inserting a fragment containing SA, the Gateway cassette (Invitrogen, Carlsbad, Calif.), polyA, and PGKneo between the 5′ and 3′ homologous arms of the ROSA 26 targeting arms.

A gene of interest, such as a secreted factor of interest, can be cloned into a master ROSA26 targeting vector, as shown in FIG. 1. The endogenous ROSA26 promoter will drive the expression of the secreted factor. The ROSA26 promoter resides in the 5′ homologous arm. The 5′ and 3′ homologous arms and the TK (HSV thymidine kinase) gene can be used to target the secreted factor to the ROSA 26 locus in the mouse ES cells. The PGKneobpA can be used as a selection marker for the targeting experiment. The SA and bpA sequences facilitate the expression of the secreted factor. The above described targeting fragment can be cloned into the multiple cloning site of a plasmid (e.g. pBluescript from Stratagene, La Jolla, Calif.). The plasmid backbone is not shown in the figure.

The master targeting vector was constructed by cloning the SA (PCR amplified) and bpA fragment into pBluescript. Then the PGKneobpA was cloned 3′ to the bpA fragment. The Gateway conversion cassette (Invitrogen, Carlsbad, Calif.) was then cloned between SA and bpA. Then the whole fragment containing the SA, Gateway cassette, polyA, and PGKneo was cloned between the 5′ and 3′ homologous arms of the ROSA 26 targeting arms (the 5′ and 3′ homologous arms were PCR amplified and cloned from genomic DNA according to the public genomic database). Only the targeted clones were demonstrated to have a PCR product.

The genes of interest, such as the secreted factor genes, can be cloned into a Gateway entry vector first and subsequently cloned into the ROSA 26 targeting vector by the Gateway cloning technology (Invitrogen, Carlsbad, Calif.).

### Example 2

### Targetability as a Screening Method for Identifying Potent Factors that Inhibit ES Cell Proliferation or Induce Differentiation

The same homologous arms as above are used for targeting all the secreted molecules to the ROSA 26 locus. The initial number of secreted molecules selected for targeting/expression is about 100-200. The ‘potent’ factors that inhibit ES cell growth or induce differentiation can be discovered by the fact that no targeted clones can be obtained solely for these clones.

### Example 3

### Test Synergistic Effects of Combinations of Secreted Factors in Proliferation and Differentiation

Different ES clones from the ES cell library were co-cultured in various combinations. A pool of cells was generated that secrete a number of secreted molecules simultaneously. Proliferation/differentiation is a result of combination of signals. A number of pools of ES cells, with each pool containing ES cells expressing up to about 10 different secreted molecules, were generated. The effects of these pools of secreted factors on differentiation of ES cells (see examples that follow) or proliferation of other cell types was then tested.

### Example 4

### Study of Secreted Factors' Function in Hematopoietic Lineages

ES cells can differentiate into mature hematopoietic cells under defined experimental conditions. For example, erythropoietin and/or interleukin I a (IL-1a) plus IL 3 can induce this differentiation. The library of ES cells, each ES clone expressing a different secreted molecules, was tested to see if any of the cell clone's ability to differentiate was altered (increase or decrease). Lineage marker(s) and morphology was used to follow differentiation. In addition, the synergistic effect of the combination of secreted molecules was tested as described in example 3.

### Example 5

### Study of Secreted Factors' Function in Pancreatic Beta Cell Differentiation

The multiple pools of ES cells generated as described in Example 3 are used to test the ability of the secreted factor combinations to induce ES cells' differentiation potential to pancreatic beta cells. A number of beta cell markers (e.g. insulin, PDX-1, PAX-4, PAX-6, Nkx2.2 and Nkx6.1, insulin I, insulin II, glucose transporter 2) are used to track the differentiation.

Accordingly, stem cell libraries and methods of using the same are disclosed. From the foregoing, it will be appreciated that, although preferred embodiments of the subject invention have been described in some detail, it is understood that obvious variations can be made without departing from the spirit and the scope of the invention as defined by the appended claims.

### Example 6

### In Vivo Use of ES Cells Containing Introduced Nucleic Acid Molecules

A pool of ES cells were transformed with a nucleic acid molecule of interest using standard techniques and maintained in culture in vitro under conditions that allow proliferation but not differentiation. Transformed ES cells were obtained from the pool and are introduced into a normal mouse blastocyst to produce a chimeric blastocyst. Typically from about 4-50 transformed ES cells, more typically, about 10-40 ES cells and particularly about 20 ES cells were injected into a blastocyst, such that about 1 out of 10 to about 6 out of 8 cells in the blastocyst represent transformed ES cells. The chimeric blastocyst can then be implanted into the uterus of a suitable surrogate female mouse for further embryonic development. One or more of such blastocysts, for example up to eight, can be implanted per surrogate female mouse. Upon birth of a chimeric mouse from a chimeric blastocyst, the chimeric mouse can be studied to determine the function of the introduced nucleic acid molecule. Standard methods implantation are known in the art. See, e.g., C. L. Stewart, p. 823 in “Methods of Enzymology” Volume 225 (P. M. Wassarman and M. L. DePamphilis, eds.) Academic Press, 1993.

Any suitable function of the introduced nucleic acid molecule can be examined using the chimeric mouse, including but not limited to, for example, ligand function or receptor function; its function in tissue morphogenesis; stimulation of differentiation, stimulation of proliferation, inhibition, or activation of different cellular systems, including: (1) hematopoietic system including T cells, B cells, NK cells, dendritic cells, monocytic, other cells of the hematopoietic system; (2) beta islet cells of the pancreas; (3) chondrocytes; (4) bone marrow system including osteoclasts, osteoblasts, and stromal cells; (5) cardiovascular system including cardiomyocytes; (6) CNS and spinal cord including neuronal cells; and so forth.

The chimeric mouse, tissues and cells thereof can be dissected for observation of their functional states, including but not limited to: number of the target cell type that is of interest, such as T cells, for example, the stage of development of the cell, whether it is mature or immature, the functional status of the cell, and the activation status of the cell, etc.

1-123. (canceled)

124. A modified mesenchymal stem cell comprising at least one first heterologous nucleic acid sequence encoding at least one first therapeutic molecule for a disease, disorder, syndrome, or condition, wherein said sequence is other than an anti-cancer agent.

125. The modified mesenchymal stem cell of claim 124, wherein the disease, disorder, syndrome, or condition is other than a hyperproliferative disease, disorder, syndrome, or condition.

126. The modified mesenchymal stem cell of claim 124, wherein the first therapeutic molecule is secreted.

127. The modified mesenchymal stem cell of 124, wherein the first therapeutic molecule is a molecule chosen from a growth factor, an anti-inflammatory factor, an anti-apoptotic factor, a vasodilator, an anti-coagulant, insulin, an extracellular domain of a receptor, an immunoglobulin chain, a biologically active fragment of the molecule, and a fusion protein comprising the molecule, or biologically active fragment thereof.

128. The modified mesenchymal stem cell of claim 127, wherein the first therapeutic molecule is a fusion protein and the fusion protein is cleavable to release a biologically active molecule or biologically active fragment thereof.

129. The modified mesenchymal stem cell of claim 124, further comprising at least one second heterologous nucleic acid sequence encoding a second therapeutic molecule that functions in conjunction with the first therapeutic molecule.

130. The modified mesenchymal stem cell of claim 127, wherein the first therapeutic molecule is a growth factor selected from a factor that stimulates proliferation of a cell, a factor that stimulates differentiation of a cell, a factor that promotes change in the metabolic state of a cell, and a factor that promotes change in the gene expression pattern of a cell.

131. The modified mesenchymal stem cell of claim 127, wherein the change in the metabolic state of the cell comprises activation.

132. The modified mesenchymal stem cell of claim 124, wherein the first therapeutic molecule is a growth factor selected from KGF, PDGF, BDNF, erythropoietin, GM-CSF, and G-CSF.

133. The modified mesenchymal stem cell of claim 127, wherein the first therapeutic molecule is an anti-inflammatory factor.

134. The modified mesenchymal stem cell of claim 133, wherein the anti-inflammatory factor is an anti-TNF molecule.

135. The modified mesenchymal stem cell of claim 133, wherein the anti-inflammatory factor is an extracellular fragment of a receptor.

136. The modified mesenchymal stem cell of claim 124, wherein the cell is selected from a CNS cell, heart muscle cell, lung cell, kidney cell, bone marrow cell, lymphocyte, lymphocyte precursor, erythrocyte precursor, bone cell, cartilage cell, pancreatic cell, liver cell, muscle cell, fibroblast, epithelial cell, and skin cell.

137. The modified mesenchymal stem cell of claim 124, further comprising at least one third heterologous nucleic acid sequence encoding a survival factor.

138. The modified mesenchymal stem cell of claim 137, wherein the survival factor is an anti-apoptotic factor.

139. The modified mesenchymal stem cell of claim 137, wherein the survival factor is Akt.

140. The modified mesenchymal stem cell of claim 137, wherein the survival factor is telomerase.

141. The modified mesenchymal stem cell of claim 124, wherein the first heterologous nucleic acid sequence is under the regulatory control of a promoter.

142. The modified mesenchymal stem cell of claim 141, wherein the promoter is an inducible promoter.

143. The modified mesenchymal stem cell of claim 141, wherein the promoter is a constitutive promoter.

144. The modified mesenchymal stem cell of claim 141, wherein the promoter is a tissue-specific promoter.

145. The modified mesenchymal stem cell of claim 141, wherein the promoter is heterologous to the first heterologous nucleic acid molecule.

146. The modified mesenchymal stem cell of claim 141, wherein the promoter is homologous to the first heterologous nucleic acid molecule.

147. A composition comprising the modified mesenchymal stem cell of claim 124 and a pharmaceutically acceptable carrier.

148. A kit comprising the composition of claim 147 and instructions for administration into an animal.

149. The kit of claim 148, wherein the animal is a human.

150. A modified mesenchymal stem cell comprising at least one first heterologous nucleic acid sequence and at least one second heterologous nucleic acid sequence, wherein the first heterologous sequence encodes one member of a dimeric molecule and the second heterologous nucleic acid encodes a second member of the dimeric molecule.

151. The modified mesenchymal stem cell of claim 150, wherein the dimeric molecule is a therapeutic molecule.

152. The modified mesenchymal stem cell of claim 150, wherein the dimeric molecule is an antibody.

153. The modified mesenchymal stem cell of claim 150, wherein the first heterologous nucleic acid sequence encodes an immunoglobulin heavy chain and the second heterologous nucleic acid sequence encodes an immunoglobulin light chain.

154. The modified mesenchymal stem cell of claim 152, wherein the antibody is an anti-inflammation antibody.

155. The modified mesenchymal stem cell of claim 152, wherein the antibody is an anti-cancer antibody.

156. A composition comprising the modified mesenchymal stem cell of claim 150 and a pharmaceutically acceptable carrier.

157. A kit comprising the composition of claim 156 and instructions for administration into an animal.

158. The kit of claim 157, wherein the animal is a human.

159. A modified mesenchymal stem cell comprising a mesenchymal stem cell that comprises at least one first heterologous nucleic acid sequence, wherein the first heterologous nucleic acid sequence encodes a therapeutic factor that is therapeutic for cancer and is other than a cytokine, a hormone, an extracellular matrix component, an enzyme, a signaling molecule, an anti-angiogenic polypeptide, an oncolytic virus, interferon-α, or interferon-β.

160. The modified mesenchymal stem cell of claim 159, wherein the therapeutic factor is an immunoglobulin.

161. The modified mesenchymal stem cell of claim 160, wherein the therapeutic factor is a single chain antibody.

162. A method for treatment of a disease, disorder, syndrome, or condition in a subject comprising the steps of:
(a) providing a modified mesenchymal stem cell of claim 124; and
(b) administering the modified mesenchymal stem cell to the subject, wherein the disease, disorder, syndrome, or condition is other than cancer.

163. The method of claim 162, wherein the disease, disorder, syndrome, or condition is other than a hyperproliferative disease, disorder, syndrome, or condition.

164. The method of claim 162, wherein the disease, disorder, syndrome, or condition is inflammation and the first therapeutic molecule is an anti-inflammatory factor.

165. The method of claim 162, wherein the disease, disorder, syndrome, or condition is ischemic heart disease and the first therapeutic molecule is a vasodilator.

166. The method of claim 162, wherein the disease, disorder, syndrome, or condition is thrombosis and the first therapeutic molecule is an anti-coagulant.

167. The method of claim 162, wherein the disease, disorder, syndrome, or condition is diabetes and the first therapeutic molecule is insulin.

168. The method of claim 162, wherein the disease, disorder, syndrome, or condition is immunological, and the first therapeutic molecule is immunosuppressive.

169. The method of claim 162, wherein the disease, disorder, syndrome, or condition is immunological, and the first therapeutic molecule enhances the immune response of the subject.

170. A method for treatment of a hyperproliferative disease, disorder, syndrome, or condition in a subject comprising the steps of:
(a) providing a modified mesenchymal stem cell of 124; and
(b) administering the modified mesenchymal stem cell to the subject, wherein the first therapeutic molecule encoded by the first heterologous nucleic acid sequence is an antibody.

171. The method of claim 170, wherein the antibody is a single chain antibody.

172. A method for treatment of a disease, disorder, syndrome, or condition in a subject comprising the steps of:
(a) providing a modified mesenchymal stem cell of claim 130; and
(b) administering the modified mesenchymal stem cell to the subject.

173. The method of claim 172, wherein the disease, disorder, syndrome, or condition is a hyperproliferative disease, disorder, syndrome, or condition.

174. The method of claim 173, wherein the hyperproliferative disease, disorder, syndrome, or condition is cancer.

175. The method of claim 173, wherein the hyperproliferative disease, disorder, syndrome, or condition is psoriasis.

176-242. (canceled)
